(19) United States
(12) Patent Application Publication
(10) Pub. No.: US 2009/0209826 A1
(43) Pub. Date: Aug. 20, 2009

(54) Intubation systems and methods

(75) Inventors: Gerald Jay Sanders, Sonoma, CA (US); Zebadiah Kimmel, Cambridge, MA (US); Raymond Glassenberg, Wilmette, IL (US); Vivek Sikri, Cambridge, MA (US)

Correspondence Address: MINTZ LEVIN COHN FERRIS GLOVSKY & POPEO, ONE FINANCIAL CENTER, BOSTON, MA, 02111, US

(73) Assignee: EZC MEDICAL LLC, San Francisco, CA (US)

(21) Appl. No.: 12/319,614

(22) Filed: Jan. 9, 2009

Related U.S. Application Data
(60) Provisional application No. 61/010,673, filed on Jan. 9, 2008.
(60) Provisional application No. 61/192,211, filed on Sep. 16, 2008.

(51) Int. Cl. A61B 1/267

(52) U.S. Cl. 600/188; 600/185

(57) ABSTRACT

A device, a system, and a method for intubation are disclosed. The intubation device includes a handle disposed at a proximal end of the intubation device, a guiding tip having a camera disposed at a distal end of the intubation device, an inter-trachea tube connecting the handle and the guiding tip. The camera is configured to guide insertion of an intra-trachea tube into a patient, wherein the guiding tip is configured to be inserted inside the intra-trachea tube while the handle remains outside the intra-trachea tube.

### CROSS-REFERENCE TO RELATED APPLICATIONS

The present application claims priority to U.S. Provisional Patent Application No. 61/010,673, filed Jan. 9, 2008, and entitled “Intubation Systems and Methods” and U.S. Provisional Patent Application No. 61/192,211, filed Sep. 16, 2008, and entitled “Intubation Device, System and Method for Intubation”, and incorporates their disclosures herein by reference in their entireties.

### BACKGROUND OF THE INVENTION

1. Field of the Invention

The present invention relates to devices, systems and methods for imaging a body tissue during medical procedures. Specifically, the present invention relates to systems and methods for intubation of a patient using a camera device for guiding an intra-trachea tube.

2. Background

Fiber optic tracheal intubation is a useful technique in patients whose tracheas are difficult to intubate. However, there are two major difficulties with this technique. The first is the location of the glottis and insertion of a fiber scope into the trachea. Induction of general anesthesia (with or without neuromuscular block) causes the soft palate, tongue and epiglottis to approximate to the posterior pharyngeal wall, and thus little air space is left in the oropharynx for maneuvering the tip of the fiber scope to locate the glottis. The second difficulty is insertion of a tube over the fiber scope into the trachea. There have been reports of failed tracheal intubation despite successful insertion of a fiber scope into the trachea.

To solve the first difficulty, several maneuvers (such as thrusting the jaw forward, extension of the head, or traction of the tongue) have been proposed, and airway intubators have been developed. In addition, there have been comprehensive articles discussing effective ways of teaching fiberscopy. In contrast, there have been no textbooks or reviews which comprehensively deal with the second difficulty, of advancing a tracheal tube over a fiber scope.

Other problems in fiber optic intubation include apnea and upper airway damage. With respect to apnea, the time taken to intubate the trachea, and thus the duration of apnea, is generally markedly longer for fiber optic intubation than intubation with a laryngoscope. As a consequence, stress responses, such as the increase in the heart rate and blood pressure, are more likely to be greater during fiber optic intubation. If apnea continues unduly, the patient may become hypoxic. It is particularly awkward if, after successful insertion of a fiber scope into the trachea despite considerable difficulty in a patient with a difficult airway, it is still difficult to advance a tube over the scope into the trachea and the arterial hemoglobin oxygen saturation starts to decrease.

With regard to damage to the upper airway, repeated attempts at inserting a fiber scope into the trachea and advancing a tube over the scope increase the risk of injury to the larynx and surrounding tissues, leading to bleeding from, or edema of, the tissues. Although rare, complete airway obstruction could occur during attempts at fiber optic intubation even when the patient is not anaesthetized. What tends to be ignored is that at no time during insertion of a tracheal tube over a fiber scope can the tip of the tube be seen directly. Therefore, a tracheal tube should be advanced over a fiber scope with great caution, particularly in patients with pathological changes to the glottis or surrounding tissues. In a patient with laryngeal papillomatosis, repeated attempts at passing a tube over a successfully inserted fiber scope into the trachea resulted in massive bleeding, necessitating an emergency surgical airway.

Thus, there is a need for a better intubation device that is capable of providing guidance when intubating a patient down patient's intra-trachea tube.

### SUMMARY OF THE INVENTION

In some embodiments, the present invention relates to an intubation device for insertion into a intra-trachea tube for guiding the intra-trachea tube into a patient that includes a handle disposed at a proximal end of the intubation device, a guiding tip having a camera disposed at a distal end of the intubation device, and an inter-trachea tube connecting the handle and the guiding tip. The camera is configured to guide insertion of the inter-trachea tube and the guiding tip are configured to be inserted inside the intra-trachea tube, while the handle remains outside the intra-trachea tube.

In some embodiments, the present invention relates to an intubation device for insertion into a intra-trachea tube of a patient that includes a handle disposed at a proximal end of the intubation device, a guiding tip having a camera disposed at a distal end of the intubation device, and a wire bundle connecting the handle and the guiding tip. The camera is configured to guide insertion of an intra-trachea tube within a patient.

In some embodiments, the present invention relates to an intubation device for insertion into a intra-trachea tube for guiding the intra-trachea tube into the patient that includes a handle disposed at a proximal end of the intubation device, a guiding tip having a camera disposed at a distal end of the intubation device, and a malleable metal tube/shaft connecting the handle and the guiding tip. The camera is configured to guide insertion of an intra-trachea tube into a patient when the intubation device is positioned within the intra-trachea tube. The guiding tip is preferably configured to be positioned inside the intra-trachea tube of the patient, while the handle remains outside the intra-trachea tube.

In some embodiments, the present invention relates to an intubation device for examination of a patient. The device includes a handle disposed at a proximal end of the intubation device, a guiding tip having a camera disposed at a distal end of the intubation device, a shaft portion connecting the handle and the guiding tip. The camera is configured to guide insertion of the shaft portion into a patient, wherein the guiding tip is configured to be inserted inside the shaft portion while the handle remains outside the patient. The shaft portion includes an articulation mechanism configured to angularly articulate position the guiding tip with respect to the shaft.

In some embodiments, the present invention relates to an intubation device for examination of a patient. The device includes a handle disposed at a proximal end of the intubation device, a guiding tip having a camera disposed at a distal end of the intubation device, and a shaft portion connecting the handle and the guiding tip. The camera is configured to guide insertion of the shaft portion into a patient, wherein the guiding tip is configured to be inserted inside the shaft portion while the handle remains outside the patient. The shaft portion includes a dilation mechanism configured to dilate an examination cavity within the patient.

In some embodiments, the present invention relates to a method of intubating a patient using an intubation device. The device includes a handle disposed at a proximal end of the intubation device, a guiding tip having a camera disposed at a distal end of the intubation device, and a shaft portion connecting the handle and the guiding tip. The shaft portion includes an articulation mechanism configured to angularly articulate position the guiding tip with respect to the shaft. The method includes the steps of using the camera, guiding insertion of the guiding tip and shaft portion into a patient, wherein the guiding tip is configured to be inserted inside the shaft portion while the handle remains outside the patient, using the articulation mechanism, angularly articulating the guiding tip within the patient, and using the camera, examining the patient.

In some embodiments, the present invention relates to a method for intubating a patient using an intubation device. The device includes a handle disposed at a proximal end of the intubation device, a guiding tip having a camera disposed at a distal end of the intubation device, and a shaft portion connecting the handle and the guiding tip. The shaft portion includes a dilation mechanism configured to dilate an examination cavity within the patient. The method includes the steps of using the camera, guiding insertion of the shaft portion into a patient, wherein the guiding tip is configured to be inserted inside the shaft portion while the handle remains outside the patient, using the dilation mechanism, dilating the examination cavity within the patient, and using the camera, examining the examination cavity.

### BRIEF DESCRIPTION OF THE DRAWINGS

The present invention is described with reference to the accompanying drawings. In the drawings, like reference numbers indicate identical or functionally similar elements. Additionally, the left-most digit(s) of a reference number identifies the drawing in which the reference number first appears.

FIGS. 1-4 illustrate an exemplary embodiment of an intubation device, according to some embodiments of the present invention.

FIGS. 5-8 illustrate another exemplary embodiment of an intubation device, according to some embodiments of the present invention.

FIGS. 9-12 illustrate yet another exemplary embodiment of an intubation device, according to some embodiments of the present invention.

FIG. 13 illustrates a portion of a tube of an intubation device, according to some embodiments of the present invention.

FIGS. 14a-b illustrate various methods of securing the intubation device to a patient, according to some embodiments of the present invention.

FIG. 15 illustrates another exemplary intubation device, according to some embodiments of the present invention.

FIG. 16 is an exploded view of the exemplary intubation device shown in FIG. 15.

FIG. 17 illustrates an exemplary tip portion of the intubation device shown in FIG. 15.

FIG. 18 illustrates an exemplary camera portion disposed in the tip portion of the intubation device shown in FIG. 15.

FIG. 19 is an exploded view of a handle portion of the intubation device shown in FIG. 15.

FIG. 20 is an exploded view of a plunger portion of the handle portion of the intubation device shown in FIG. 15.

FIG. 21 is another exemplary intubation device, according to some embodiments of the present invention.

FIG. 22 is a cross-sectional view of the handle portion of the intubation device shown in FIG. 15.

FIG. 23 illustrates another exemplary tip portion of the intubation device, according to some embodiments of the present invention.

FIG. 24 illustrates yet another exemplary tip portion of the intubation device, according to some embodiments of the present invention.

FIG. 25 illustrate yet another exemplary intubation device, according to some embodiments of the present invention.

FIG. 26 is a cross-sectional view of the intubation device shown in FIG. 25.

FIG. 27 illustrates an endo-tracheal tube being used in conjunction with the intubation device, according to some embodiments of the present invention.

FIG. 28 illustrates a directional tube, according to some embodiments of the present invention.

FIGS. 29-30 illustrate exemplary locking of the endo-tracheal tube, shown in FIG. 27, to a handle of the intubation device, shown in FIG. 25.

FIGS. 31-32 are various views of the handle of the intubation device, shown in FIG. 25.

### DETAILED DESCRIPTION OF THE INVENTION

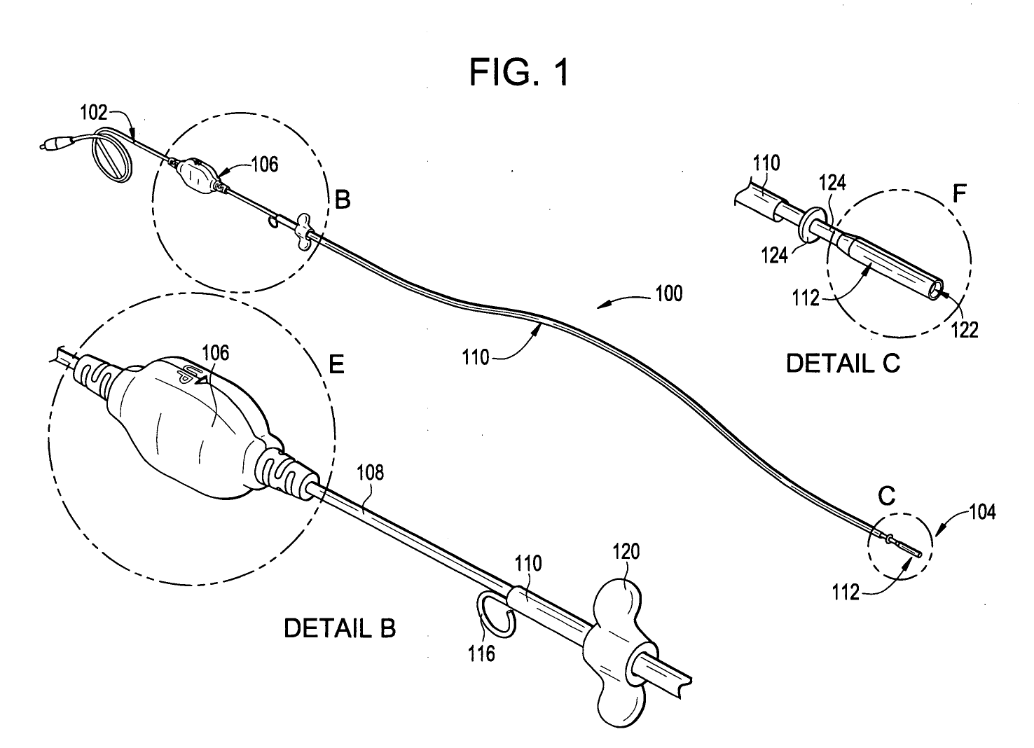

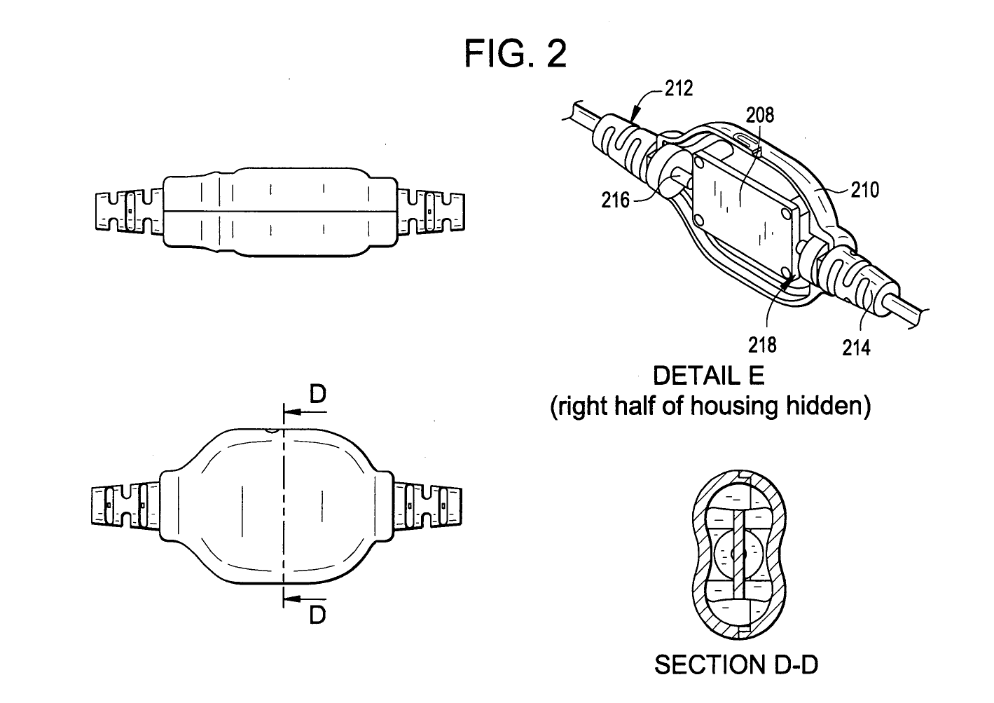

FIGS. 1-4 illustrate an exemplary embodiment of an intubation device 100. Specifically, FIG. 1 illustrates the device 100 including a Detail B excerpt and a Detail C excerpt. The device 100 is configured to be connected to an electronic equipment (not shown in FIGS. 1-4) that can supply power to and analyze data (e.g., images) received from the device 100 at a proximal end 102. The electronic equipment can include, but is not limited to, a computer or any other processing device (such as, a personal computer, a cellular telephone, a PDA, an iPod, or any other suitable device), a power supply device, a monitor, or any other device, including a combination of devices. At the proximal end 102, the device 100 includes a handle 106 that is configured assist a user (such as a doctor or any other medical professional) in guiding a tip 112, disposed at a distal end 104 of the device 100, down an intra-trachea tube 110 for insertion of the device 100 into a patient. The structure of the handle 106 is further illustrated in FIG. 2.

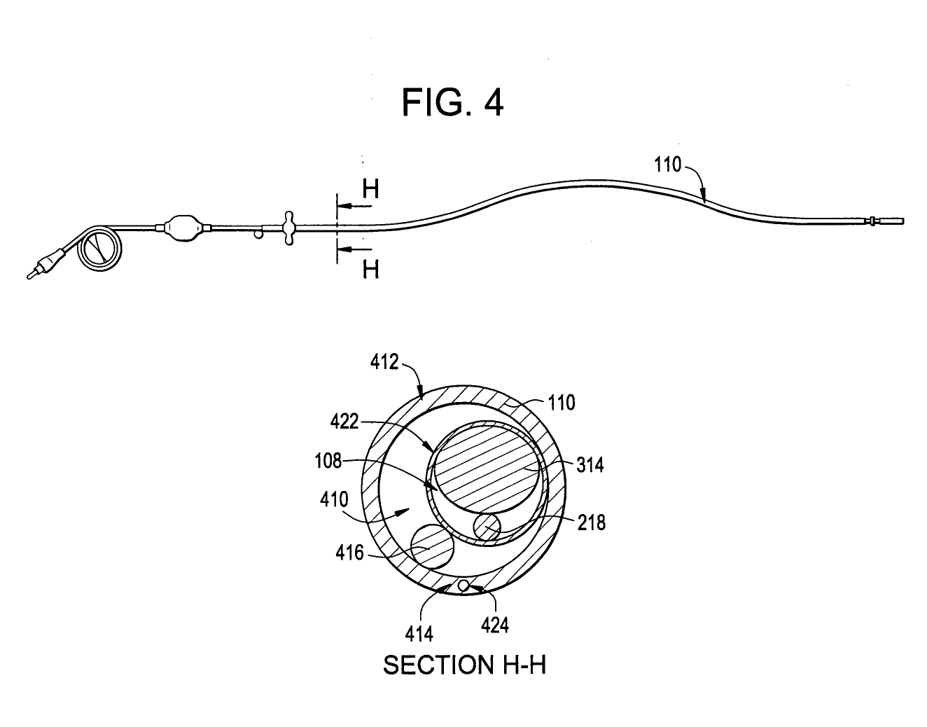

The handle 106 is further connected to a wire bundle 108 that is configured to connect the proximal end 102 and the distal end 104. The wire bundle 108 is configured to conceal a push wire (e.g., a rigid wire, not shown in FIG. 1) for advancing the distal end 104 down the intra-trachea tube, electronics cables (such as a video cable that is coupled to a camera disposed at the distal end 104), and additional channels that can be used for various purposes, including cleaning of the camera lens. The wire bundle 108 is configured to be also disposed inside an inter-trachea tube 110. The tube 110 is also disposed between the proximal end 102 and the distal end 104. In some embodiments, the tube 110 also accommodates a bendable stylette 116. The bendable stylette is configured to allow the tube 110 to be bent at particular points and further configured to hold a specific position to provide stiffness to the tube 110. The stylette 116 is also coupled to the pull wire 118 that is disposed inside a wall of the tube 110, as illustrated in FIG. 4.

The device 100 also includes an articulation handle 120 that is configured to be disposed on the tube 110 substantially adjacent the proximal end 102. The handle 120 is configured to connect to the pull wire 118 to further secure it. The handle 120 is configured to control movement of the pull wire 118 inside the tube. The user (e.g., a doctor or any other medical professional) can release the handle 120 and allow the pull wire 118 to move inside the tube 110 thereby advancing the distal end 104 in a desired direction and/or to a desired location. Upon achieving such direction/location, the user can secure the handle 120 thereby locking the wire 118 in an appropriate position.

The tip 112 is disposed at the distal end 104. The distal end 104 is configured to be inserted into an intra-trachea tube which is then guided down the throat of the patient. The tip 112 is further configured to include a camera 122 that assists the user in guiding the tip 112 not only down the intra-trachea tube of the patient, but allowing accurate guidance of the intra-trachea tube into the airways of a patient. The camera 122 is also configured to provide a view to the user of a cavity inside the patient into which the distal end 104 is inserted. The user can observe the cavity on a monitor coupled to the device 100 at a proximal end 104. The wire bundle 108 further connects to the camera 122. In some embodiments, the wire bundle 108 can include a steel wire (e.g., in some embodiments, approximately ¼ mm in diameter) for strength and a thin insulated wire, which can be bundled together for added strength. The wires allow the camera 122 to move back and forth toward the distal end 104. This allows injection of therapeutic fluid, vacuuming of fluids, etc. within the inter-trachea tube which houses the wire-bundle. In some embodiments, additional channels inside the wire bundle tube can be provided for delivery of these fluids and removal of used fluids.

The inter-trachea tube 116 may be further configured to stretch to accommodate the inter-trachea tube alignment ring 124, or the ring is flexible enough to accommodate the inter-trachea tube. The ring 124 may be disposed between the end of the inter-trachea tube 108 and the tip 112. The wire bundle 108 is configured to pass through the ring 124 to the tip 112.

FIG. 2 illustrates an interior of the handle 106 (including perspective, top, side, and cross-sectional views) along with a detailed view E from FIG. 1 and a cross-sectional view D-D. In some embodiments, the handle 106 is configured to have an irregular shape. As can be understood by one skilled in the art, the handle 106 can have many different shapes, including square, rectangular, oval, round, etc. In some embodiments, the thickness of the handle 106 can be approximately on the order of 16 mm. The width of the handle 106 can be approximately on the order 28 mm. In some embodiments, the handle 106 is configured to include two strain relief grommets 212 and 214 disposed on each side of the handle 106, as shown in FIG. 2. The length of the handle 106 with grommets 212, 214 can be on the order of approximately 69 mm, whereas without the grommets, the length of the handle 106 can be on the order of approximately 44 mm. In the cross-section, the handle 106 can also have an irregular shape (e.g., a peanut-like shape, as shown in FIG. 2). As can be understood by one skilled in the art, the handle 106 can have any other variable dimensions that can be suited for a particular purposes.

The handle 106 includes a housing 210 and a printed circuit board (“PCB”) 208. The PCB 208 is disposed inside the housing 210. The PCB 208 is further connected to a power cord 216. A stiffening wire 218 is connected to the housing 210. The power cord 216 is configured to pass through the grommet 212 and connect to a power supply equipment (not shown) to which the device 100 connects to receive power. In some embodiments, the power cord 216 is configured to provide power coming in and video signal going out. The power cord is further configured to be coupled to a plug or any other type of connector disposed at a proximate end 102 (shown in FIG. 1). The plug is configured to be connected to a monitor, a computer, or any other processing device (e.g., a PC, a laptop, a cellular telephone, a PDA, an iPod, etc.) that is configured to provide power and allow the user to observe the cavity (into which the camera disposed at the distal end 104 is inserted). In some embodiments, the power cord can be used with an adapter cable that provides power from, for example, a wall outlet and sends the video to a standard monitor.

In some embodiments, the stiffening wire 218 passes through the grommet 214 outside the handle 106 and is configured to be a part of the wire bundle 108. As stated above, the stiffening wire can be ¼ mm in diameter and provide strength to the device 100. In some embodiments, the stiffening wire 218 can be manufactured from any type of metal, such as steel, or any other suitable material. The stiffening wire 218 provides rigidity to the wire bundle 108 and the intra-trachea tube 110, so that upon insertion of the device, the bundle 108 and tube 110 do not bend in an undesired ways and instead are guided toward a particular location/cavity within the patient.

As stated above, the PCB 208 is disposed inside the housing of the handle 106 and can be configured to have a thickness on the order of ½ inches. As can be understood by one skilled in the art, the PCB 208 can have any desired thickness. The above example is provided here for purely illustrative purposes. The PCB 208 is secured to the housing of the handle 106 using glue, screws, bolts, or any other suitable securing mechanism. In some embodiments, the PCB 208 can be configured to include a micro-controller and a non-volatile flash memory. The PCB 208 can also be configured to connect to a display device (not shown) via the power cord 216 and transmit signals received from the camera 122 (not shown in FIG. 2) disposed at the distal tip 104 to the display device in order to allow the user to guide an intra-trachea tube down a patient's throat.

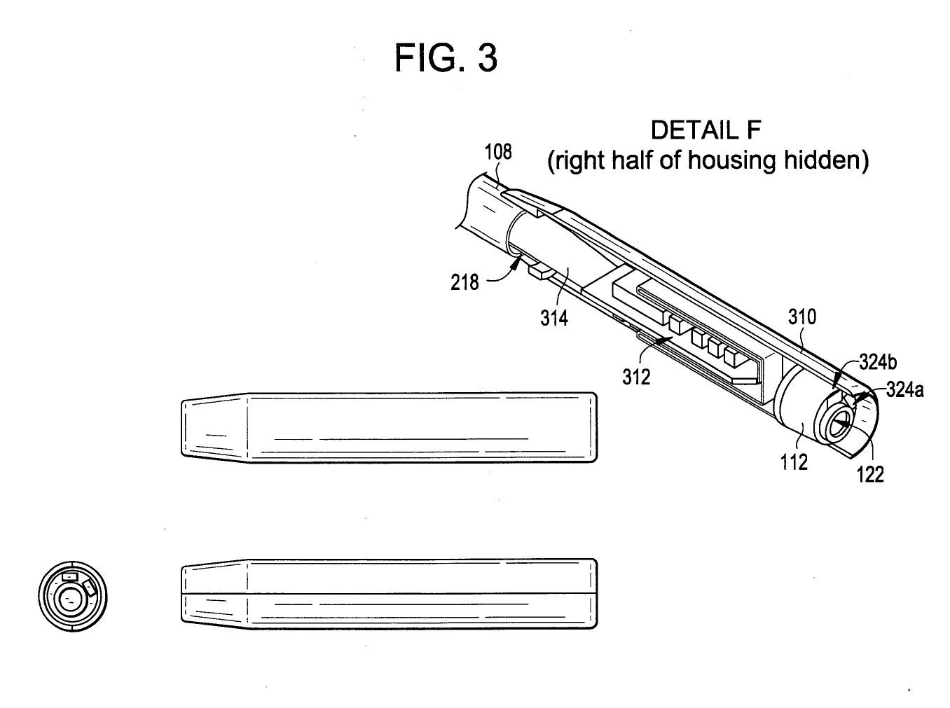

FIG. 3 illustrates in an exemplary (including cross-sectional perspective, top, and side views) the tip 112, according to some embodiments of the present invention. The tip 112 includes a housing 310. The housing 310 includes a PCB 312 that is configured to be coupled to a power/video wire 314, a stiffening wire 218, and the camera 122. The PCB 312 is configured to communicate with the PCB 208 of the handle 106 and provide video signals to the PCB 208 via power/video wire 314. The power/video wire 314 can be configured to stretch through the wire bundle 108 and connect to the PCB 208 of the handle 106 (not shown in FIG. 3). The PCB 312 and the camera 122 are configured to receive power via the power/video wire 314. Upon insertion of the tip 112 into the patient cavity, the user can activate the camera 122 (e.g., by flipping an “on” switch on a power apparatus connected to the device 100). Once the camera is activated, it begins transmitting images (e.g., still images and/or a live feed) to the PCB 312, which processes them and then further transmits them to PCB 208 via the wire 314. The PCB 208 in turn transmits such images to the processing device/monitor coupled to the proximate end 102. As stated above, the camera 122 can be configured to assist the user in guiding the camera 122 down the patient's trachea as well as observe the interior of a patient's cavity. In some embodiments, the images can be transmitted via the video portion of the wire bundle 108 and the power is supplied via a power wire of the wire bundle 108. In some embodiments, a single wire can be configured transmit image and provide power. In some embodiments, the PCB 208 and the PCB 312 can be configured to communicate wirelessly with each other as well as with the processing device/monitor coupled to the proximate end 102.

The housing 310 is configured to be coupled to the stiffening wire 218 that runs through the wire bundle 108 along with the power/video wire 314. The stiffening wire 218 allows various movement of the tip 112, including pivoting, partial and/or full rotation. The wire 218 can also be configured to cause translational and/or rotational movement of the camera 122 inside the housing 310 of the tip 112. For example, the camera 122 can be configured, using the wire 218, to be advanced toward the distal end of the tip 112 from the interior of the housing 310. Likewise, the camera can be configured, using the wire 218, to be retracted back into the housing 310.

In some embodiments, the housing 310 also includes a lighting mechanism 322 that is configured to illuminate the path inside the patient's trachea or the cavity for the camera 122. Illumination of the path and/or cavity assists the user (e.g., a doctor and/or other medical professional) in guiding the tip 112 inside the patient using the stiffening wire 218 and the handle 106. In some embodiments, the lighting mechanism 322 can include at least one light emitting diode (“LED”) 324 (a, b) that can be disposed around the camera 122 in a concentric arrangement. As can be understood by one skilled in the art, there can be more than one LED 324 disposed in the housing 310, wherein such LEDs can be disposed anywhere in the housing 310 so that they can illuminate the path for the user. The LED 324 can be configured to be coupled to the power wire 314 and the user can selectively activate them during insertion of the device 100. To prevent overheating of the housing 310 and the components disposed inside as a result of the operation of the LEDs 324, the tip 112 can be configured to include a cooling mechanism (e.g., a Peltier element) that absorbs heat generated by the LEDs 324.

In some embodiments, the camera 122 can be configured to be any conventionally known camera that can include a camera lens, image sensor(s), charge coupled device(s), etc. As can be understood by one skilled in the art, the camera 122 can be any camera suitable for the purposes of the present invention.

In some embodiments, the housing 310 can also include a protective screen 332 that can be disposed at the distal tip of the housing 310 and substantially adjacent to the camera 112. The screen can be configured to protect the camera and other components from damage. The screen 332 can be manufactured from a clear material, such as glass, Plexiglas, or any other suitable material. In some embodiments, the protective screen 332 and the camera 122 can be configured to be washed using the washing liquids supplied to the tip 112.

In some embodiments, the tip 112 can be configured to be manufactured from multiple parts and then assembled into a unitary structure, i.e., the camera 122, the LEDs 324, and the PCB 322 can be inserted into the housing 310 and then the housing 310 can secure these components. In some embodiments, the tip 112 can be configured to be manufactured as a unitary piece with its various components already integrated into it. In some exemplary embodiments, the length of the tip 112 can be approximately on the order of 28 mm and the diameter of the tip 112 can be approximately on the order of 2.25 mm. As can be understood by one skilled in the art, the tip can have any other suitable dimensions and is not limited to the above referenced proportions.

FIG. 4 illustrates perspective and cross-sectional views (taken at line H-H) of the intra-trachea tube 110. As shown in the cross-sectional view, the tube 110 is configured to include a wall 412 that encloses an interior 410. The interior 410 further encloses the wire bundle 108 (which in turn encloses the power/video/data wire 314 and the stiffening wire 218) and a bendable stylette 416 that is coupled to the bendable stylette handle 116 (shown in FIG. 1). In some embodiments, the stylette 416 is configured to allow bending of the intra-trachea tube 110 along with the components disposed inside its interior 410. In some embodiments, the wire bundle 108 can be configured to be enclosed in a protective sheath 422 to prevent damage to the power/video/data wire 314 and the stiffening wire 218.

In some embodiments, the wall 412 of the tube 110 is configured to include a lumen 414 that accommodates the pull-wire 424. One of the purposes of the pull-wire 424 is to provide a means of articulating the tip of the intubation device 100. In some embodiments, the pull-wire 424 causes intubation device 100 to be more rigid in some portions and hence, less “floppy” during operation of the device 100. Additionally, since the pull-wire 424 is placed inside the wall 412 of the tube 110, it can be configured to consume the least amount of space and as such, does not take up any space in the main internal lumen of the shaft.

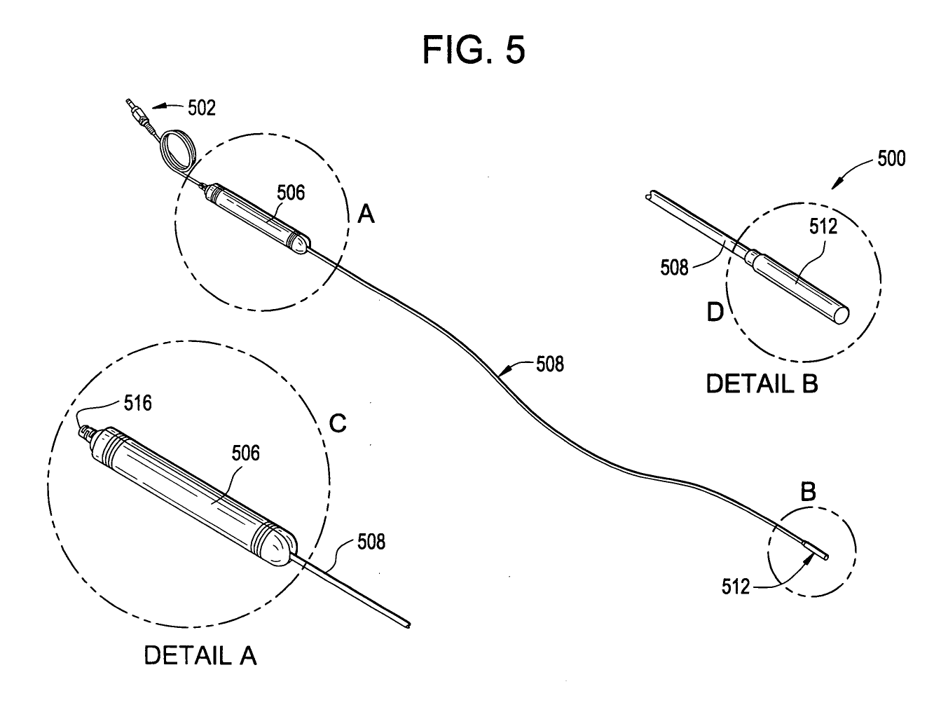

FIGS. 5-8 illustrate an alternate embodiment of the intubation device 500, according to some embodiments of the present invention. The device 500 includes a handle 506, a wire bundle 508, and a tip 512. The device 500 does not include a tube similar to the tube 110 in the embodiment shown in FIG. 1-4. Other components of the embodiment shown in FIGS. 5-8 are similar to the like components of the embodiment shown in FIGS. 1-4. FIG. 5 illustrates a perspective view of the exemplary device 500 along with detailed views A and B of the handle 500 and the tip 512, respectively. The wire bundle 508 is configured to connect the handle 506 and the tip 512. The handle 506 is configured to be coupled to a processing device/power supply via a power cord 516 in a similar fashion as the device 100 shown in FIGS. 1-4.

In the shown embodiments, the handle 506 is configured to have an elongated ellipsoidal shape. In some exemplary embodiments, the length of the handle 506 can be approximately on the order of 158 mm and the diameter of the handle can be approximately on the order of 10 mm. As can be understood by one skilled in the art, the handle 506 can have any other suitable dimensions.

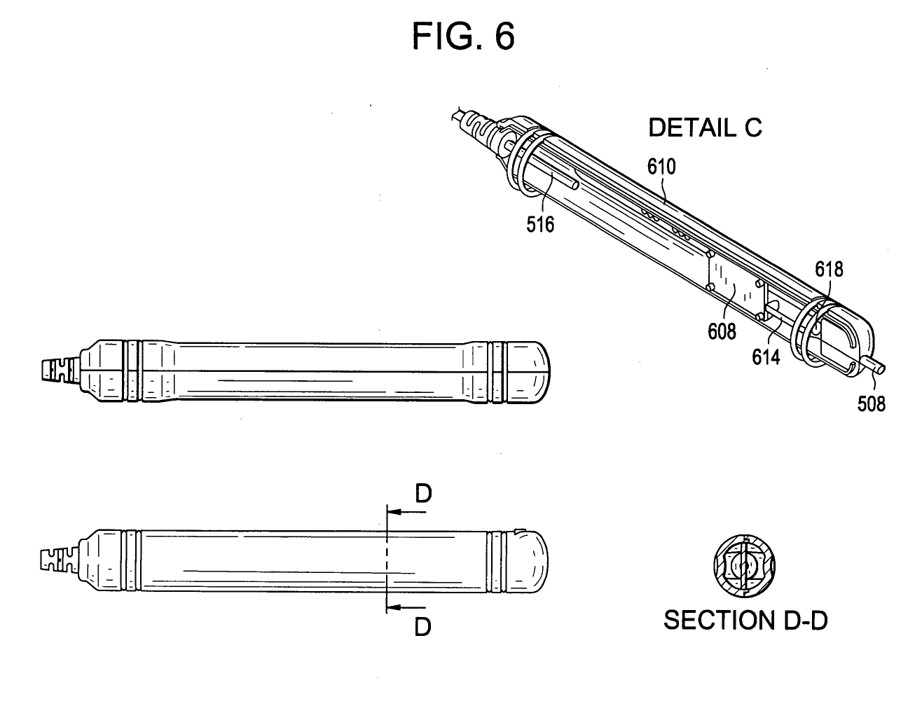

As illustrated in FIG. 6 (showing a detailed perspective cross-sectional view of an insert C in FIG. 5, top and side views, and a cross-sectional view taken along line D-D of the handle), the handle 506 includes a housing 610. The housing 610 encloses a PCB 608 that is configured to be coupled to a power cord 516 and a power/data wire 614. Along with a forming/stiffening wire 618, the wire 614 forms a wire bundle 508 in a similar fashion as shown and discussed with respect to FIGS. 1-4. In some embodiments, the diameter of the wire can be on the order of 2 mm.

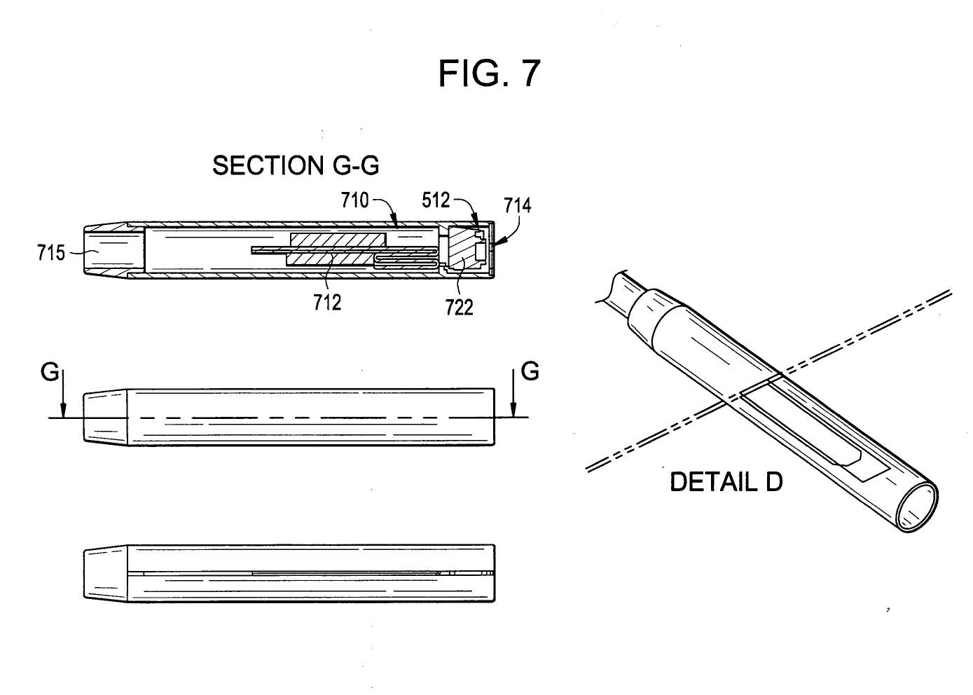

FIG. 7 illustrates detailed views (i.e., a perspective view of the tip 512 shown in detail D of FIG. 5, a cross-sectional view taken along line G-G of the tip 512 and side views) of the tip 512. The tip 512 includes a housing 710 that encloses a PCB 712 and a camera 722. Similar to the embodiment shown in FIGS. 1-4, the PCB 712 is configured to communicate with the PCB 608 disposed in the housing 610 of the handle 506. The communication is accomplished via the wire 614 that is coupled to both PCBs 608 and 712. The housing 710 further includes an opening 715, which is configured to receive the wire bundle 508. The PCB 712 is configured to communicate signals from the camera 722 to the PCB 608 disposed inside handle 506. These signals are processed and then displayed on a screen to assist the user in guiding the intra-trachea tube into a patient. In some embodiments, the tip 512 can have a length on the order of approximately 38 mm. In some embodiments, the housing 722 can also include a protective lens or a cover 714 that prevents damage to the camera 722.

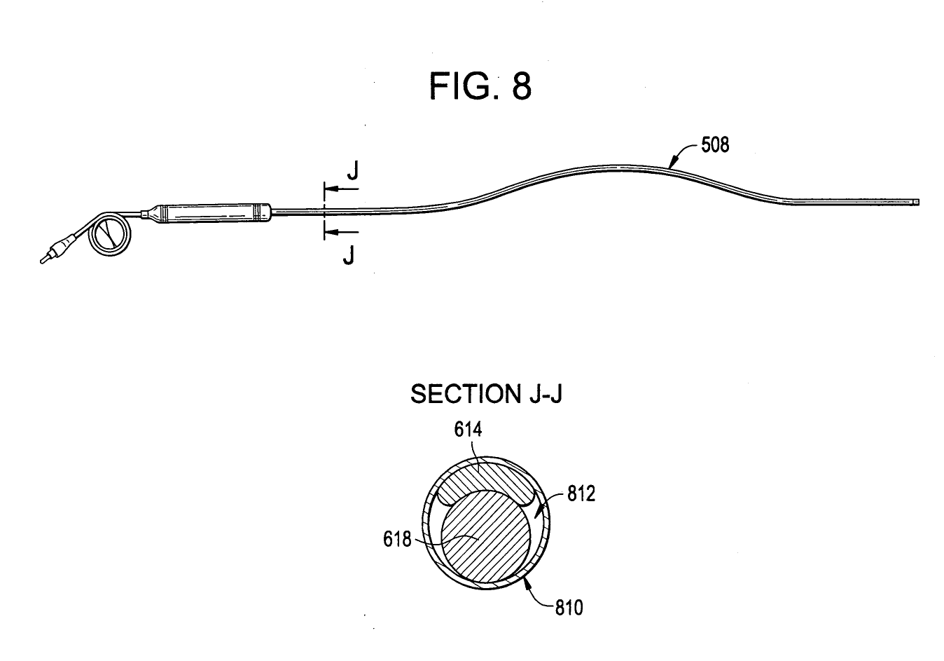

FIG. 8 illustrates perspective and cross-sectional views (taken at line J-J) of the wire bundle 508. As shown in the cross-sectional view, the bundle 508 is configured to include a wall or a sheath 810 that encloses an interior 812. The interior 812 further encloses the power/video/data wire 614 and the stiffening wire 618. In some embodiments, the device 500 can be used without the intra-trachea tube (similar to the one shown in FIG. 1). This can allow for further maneuverability and bendability of the device 500 during insertion. Further, the wire 614 can be flattened out inside the wire bundle, which also adds to the ability to maneuver the device 500 inside the patient. In other respects the device 500 is similar to the device 100 shown and discussed with regard to FIGS. 1-4.

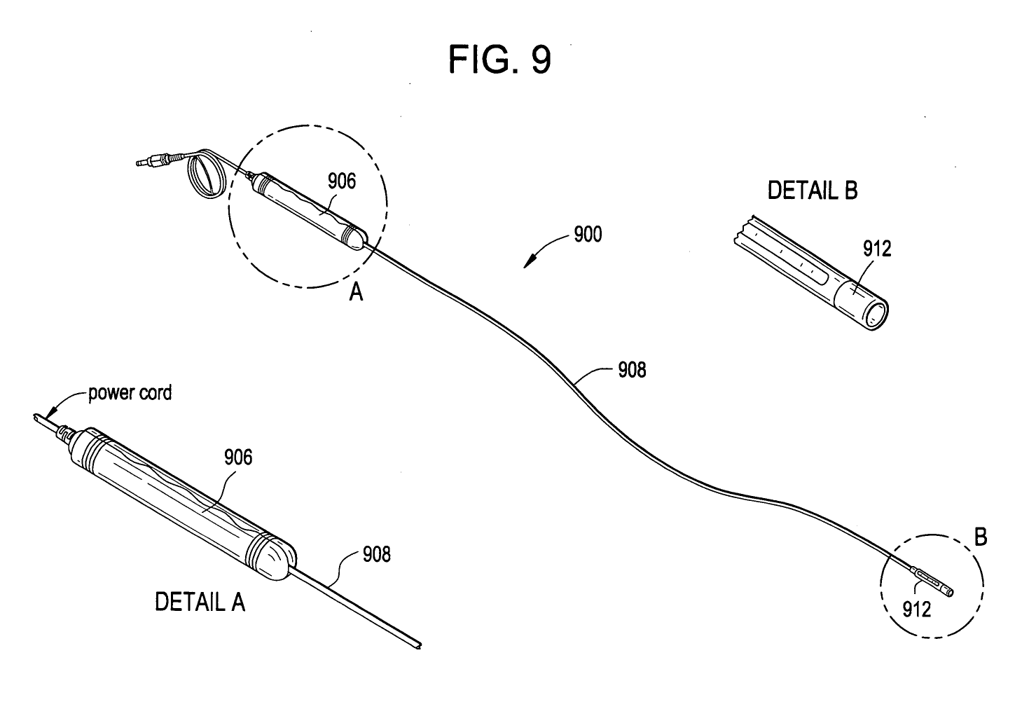

FIGS. 9-12 illustrate another exemplary intubation device 900, according to some embodiments of the present invention. The device 900 is similar to the devices 100 and 500 shown and discussed with regard to FIGS. 1-8. Thus, the following discussion focuses on elements of the device 900 that are not similar to the like elements of the embodiments shown in FIG. 1-8.

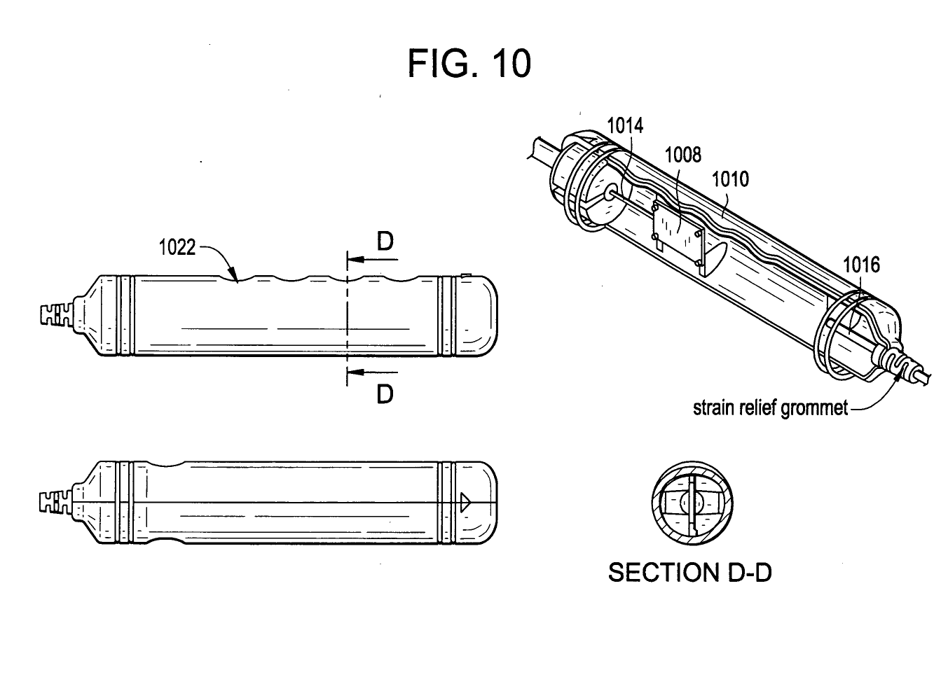

The device 900 includes a handle 906, a tip 912, and a malleable metal tube 908. The metal shaft 908 is configured to connect the handle 906 and the tip 912. As illustrated in FIG. 10, the handle 906 includes a housing 1010. The housing 1010 includes a PCB 1008 coupled to a power/data wire 1014, and a power cord 1016. In some embodiments, the handle 906 can configured to include a plurality of grips 1022, as illustrated in FIG. 10. This allows a user to firmly grip 1022 when guiding the device 900 down the intra-trachea tube, and may also allow guidance of the intra-trachea tube into the patient.

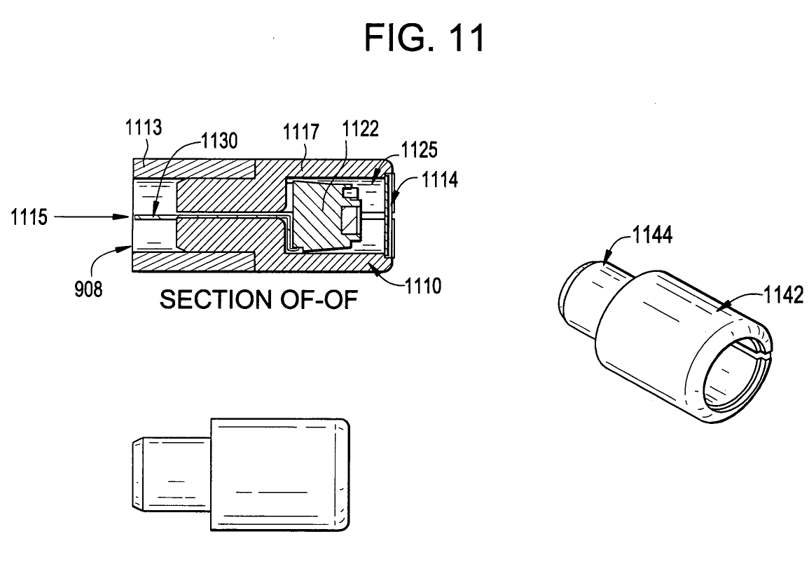

FIG. 11 is a more detailed view (including a perspective, cross-sectional, and side views) of the tip 912 of the device 900. The tip 912 includes a tip housing 1110 that is coupled to the malleable metal tube 908. The tube 908 is further configured to contain camera/data/power wire 1115. The tip housing 1110 further encloses a camera 1122 that is disposed within a hollow interior protected by the walls of the housing 1110 and a protective lens 1114, which is secured by the housing 1110. The lens 1114 is configured to protect camera 1122 from damage during insertion of the device 900 into the patient.

The housing 1110 can include a narrower portion 1113 that is configured to fit inside the metal tube 908 and a camera housing portion 1117. The camera housing portion 1117 is configured to be coupled to the narrower portion 1113. In some embodiments, the portion 1117 can be configured to be sized similarly as the tube 908, as illustrated in FIG. 11. The camera 1122 is configured to be disposed inside the camera housing portion or tip 1117. The portion 1117 further includes a hollow interior 1125 within which the camera 1122 is housed. The portion 1117 further includes a channel 1130 through which the camera wire 1115 is configured to protrude into the hollow interior 1125 for the purposes of connecting to the camera 1122. In some embodiments, the hollow interior 1125 can be configured to include LED lights similar to the LED lights shown and discussed in connection with FIGS. 1-4. The camera 1122 is configured to move inside the hollow interior 1125, once the camera tip is inserted into the tube 908.

As shown in the perspective view of the FIG. 11, in some embodiments, the camera tip 1117 can be configured to include a larger portion 1142 for housing the hollow interior 1125 and a smaller portion 1144. In some embodiments, the portions 1142 and 1144 can be integral with one another. The smaller portion 1144 is configured to fit inside and to be secured to the tube 908. Since, the sizes of the tube 908 and the larger portion 1142 are substantially similar, the transition between the tube 908 and the camera tip 1117 is substantially seamless. This feature of the present invention can be advantageous when the device 900 is inserted into the patient, as it allows for a smoother insertion of the device 900.

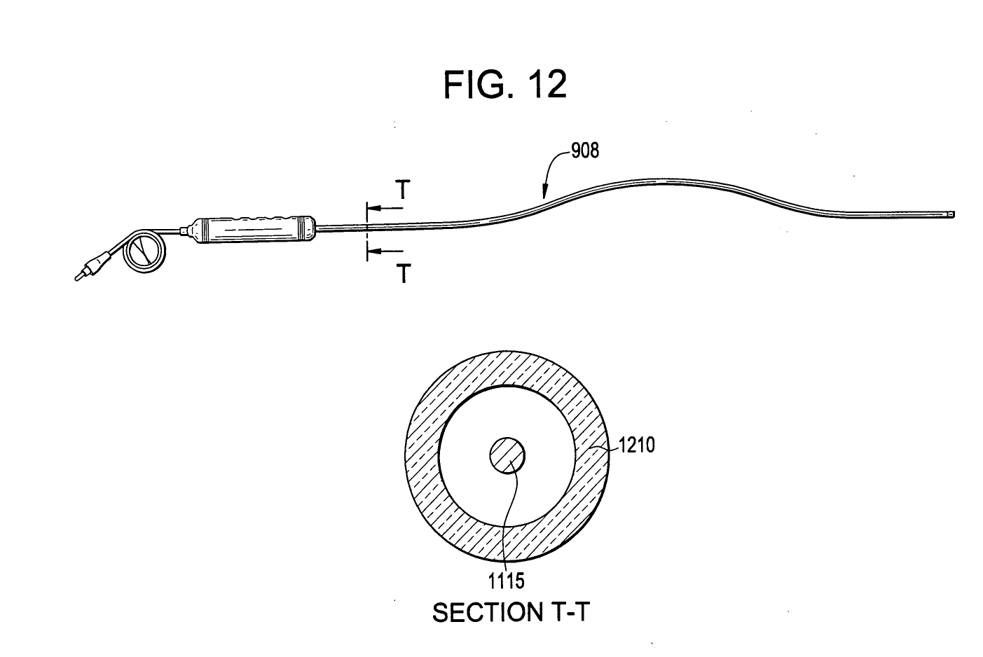

FIG. 12 illustrates a cross-section of the metal tube 908. As shown, the metal tube 908 is configured to have a wall 1210 that encloses the camera/power/data wire 1115. As can be understood by one skilled in the art, the tube 908 can be manufactured from any other materials, including, plastic, metal, polyethylene, polymer, or any other suitable material.

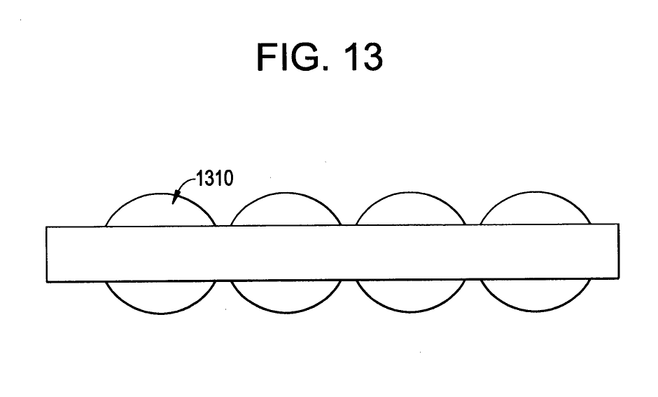

In some embodiments, the tube 110 (shown in FIGS. 1-4), the wire bundle 508 (shown in FIG. 5-8), and the malleable tube 908 (shown in FIGS. 9-12) include a plurality of protrusions 1310 configured to extend away from the surface of the above tubes, as illustrated in FIG. 13. Such protrusions 1310 are configured to secure the tube inside the intra-tracheal tube. In some embodiments, the protrusions 1310 can have an outer diameter of approximately 5 mm and be disposed along the entire length of the tube (110, 508, 908). In some embodiments, the protrusions are configured to hold these tubes centered inside the intra-trachea tube. This prevents unnecessary twisting, bending, and bundling of the tubes (110, 508, 908) when guiding the camera.

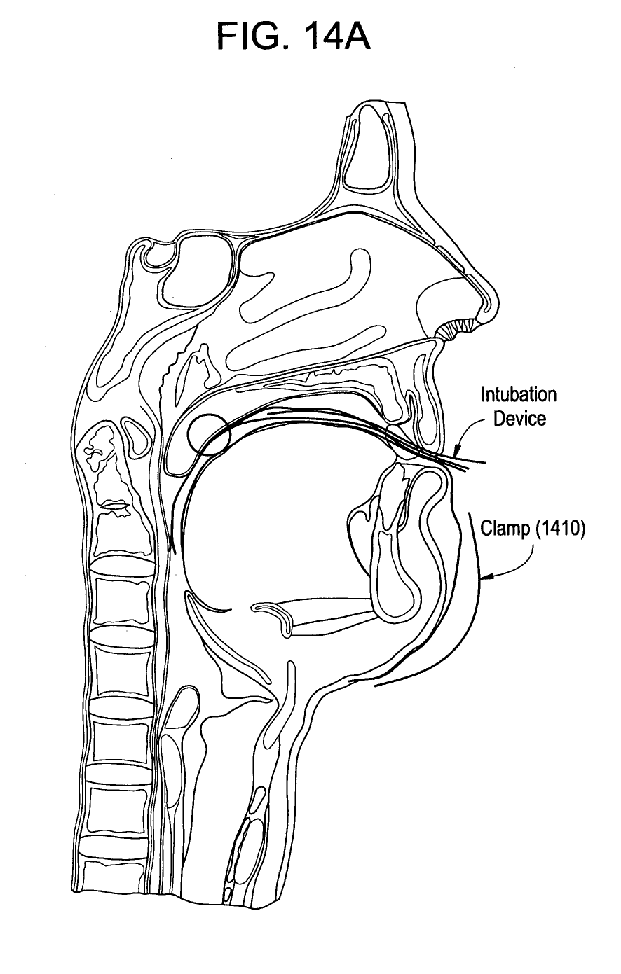

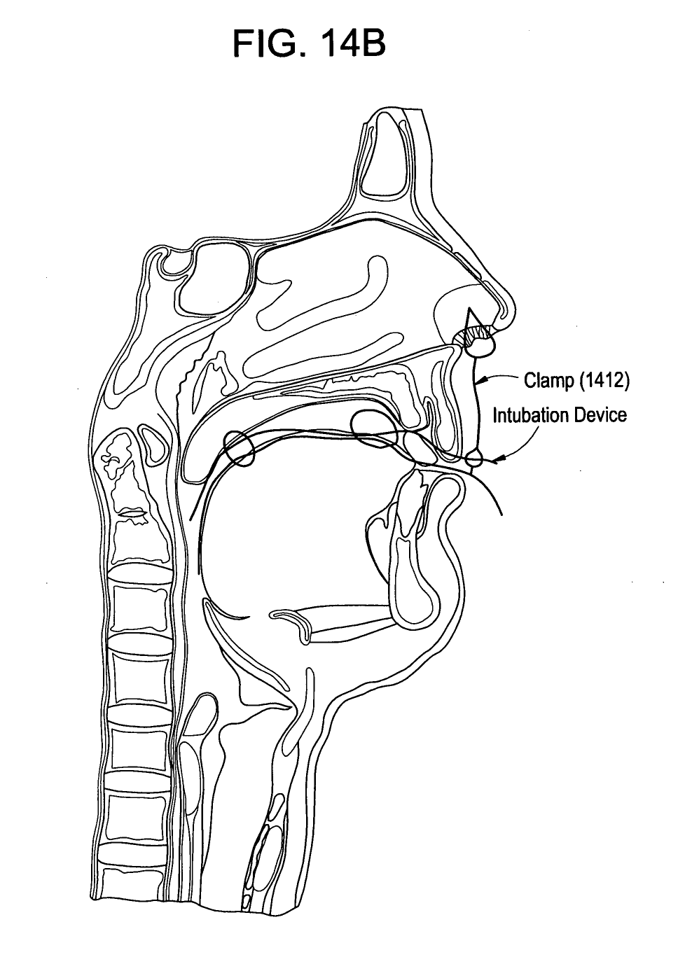

FIGS. 14a-b further illustrate how the present invention's intubation device (shown in light lines) can be inserted with a plastic clamp 1410 or a mouth clamp 1412. The clamp 1410 is configured to secure to the patient's chin and then run over the patient's tongue to be further secured inside the patient's throat, as illustrated in FIG. 14a. The clamp 1412 is configured to be secured to the patient's nose and the patient's throat, as illustrated in FIG. 14b. The clamps 1410 and 1412 include a plurality of extended section that are configured to secure an open passageway to the patient's intra-trachea tube so that the intubation device can be inserted without any obstacles.

In some embodiments, the distal end of the intubation device can be washed with saline or any other washing liquid. The camera can be moved back and forth inside the housing of the tip of the device. The housing of the tip can be filled with washing liquid, and may also include a diaphragm having a material to “wipe” the lens. Accordingly, the camera can be washed by rotating, spinning, or any other motion that allows cleaning of the camera lens using the diaphragm, with or without the use of the washing liquid (e.g., saline). Once washed, the camera can be pushed back toward the end of the tip.

In some embodiments, the present invention can include an electronic means configured to disable the device after a certain number of uses, where a “use” can be defined as turning the device from the OFF state to the ON state. In some embodiments, a non-volatile memory can be incorporated in the PCB that stores these “uses”. When device is powered up, the microcontroller reads a stored value of “uses” in the memory. In some embodiments, such memory can be a use-counter configured to increment number of “uses” after each use. Thus, after powering up, the microcontroller increments the use-counter and then stores the value to the non-volatile memory (in some embodiments, the new “use” value replaces the old “use” value). After the counter reaches a predetermined number of uses (e.g., a threshold “use” value), a signal can be generated informing the microcontroller (or any other component) to disable or turn off the device.

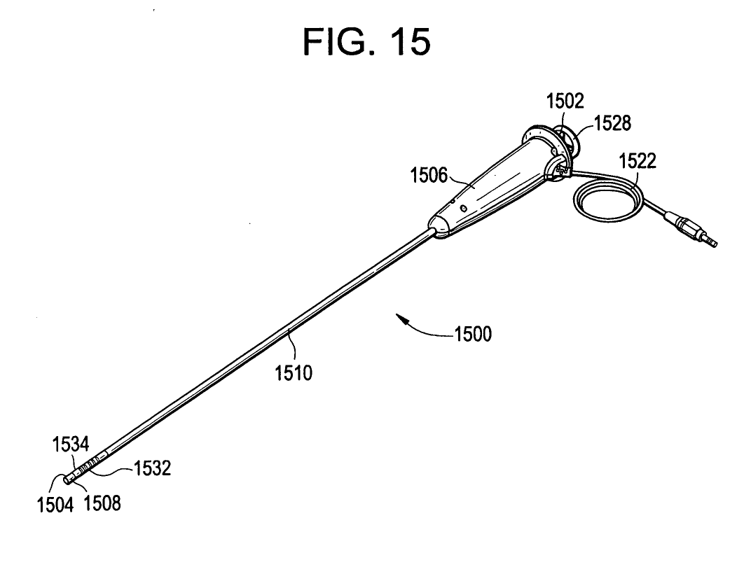

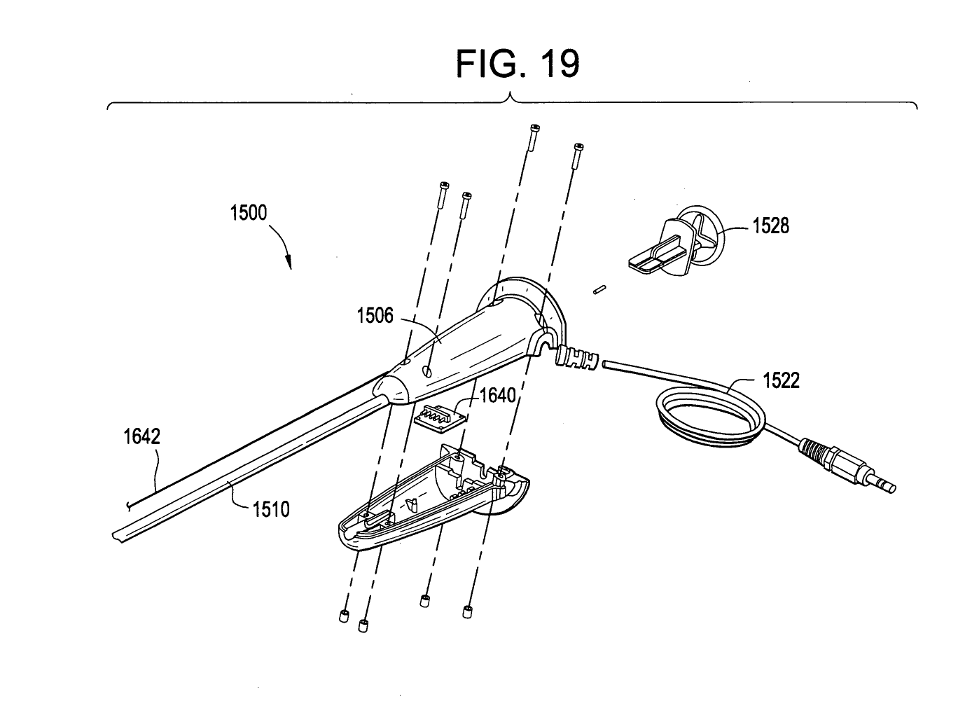

FIGS. 15-22 illustrate another exemplary embodiment of an intubation device 1500, according to some embodiments of the present invention.

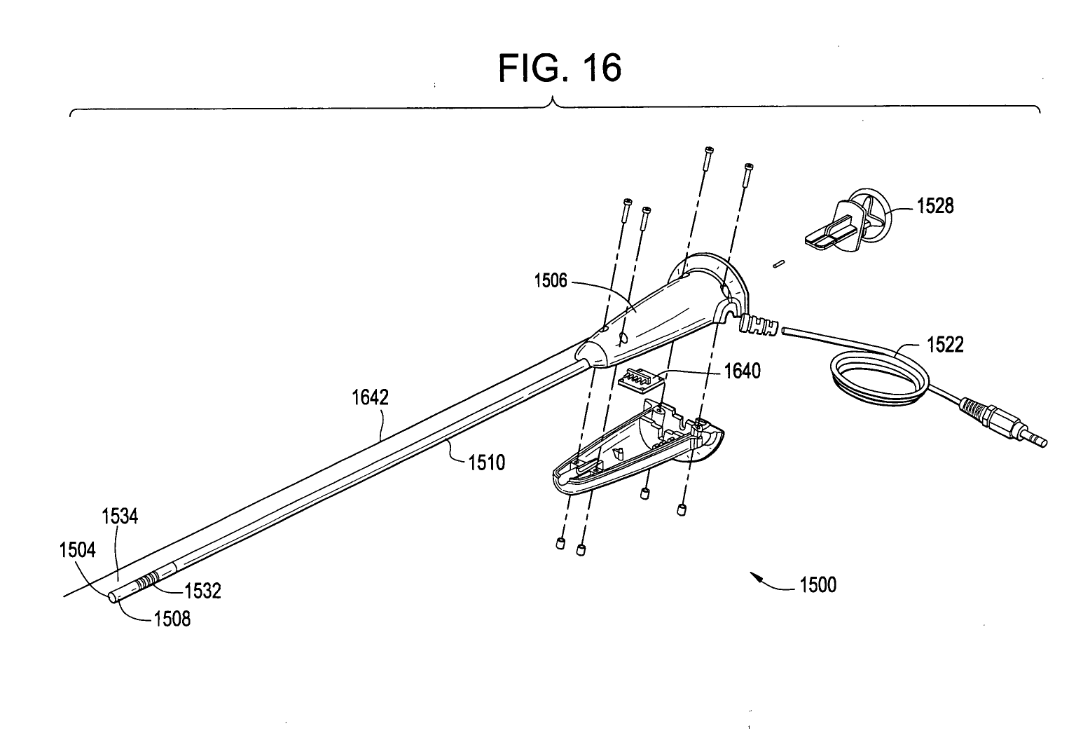

FIGS. 15 and 16 illustrate an exemplary intubation device 1500, according to some embodiments of the present invention. The device 1500 includes a proximal end 1502, a distal end 1504, a handle 1506 disposed at the proximal end 1502, and a tip 1508 disposed at the distal end 1504, and a shaft 1510 disposed between the handle 1506 and the tip 1508. In some embodiments, the shaft 1510 can be less flexible than the tip 1508. In other embodiments, the shaft 1510 and the tip 1508 can have varying degrees of flexibility. The tip 1508 can be articulated using a push wire 1642 that is coupled to the plunger portion 1528.

The tip 1508 includes a camera and an illumination unit (not shown in FIGS. 15 and 16). The camera and the illumination unit are electrically coupled to a printed circuit board (“PCB”) 1640 (as shown in FIG. 16) disposed inside the handle 1506 via wiring disposed within the shaft 1510. In some embodiments, the illumination unit can be similar to the lighting mechanism (consisting of at least one LED) of the intubation devices shown and discussed with regard to FIGS. 1-12 above. In some embodiments, the PCB 1640 can be similar to the PCB disposed inside a handle of intubation devices shown and discussed with regard to FIGS. 1-12. The PCB 1640 can further include an internal power supply device or alternatively a power supply can be disposed outside the device 1500. The camera can include a lens and a recorder unit for capturing images of a cavity to be examined using the device 1500. The recorder unit is configured to transfer captured images to the PCB 1640. The PCB 1640 is coupled to a video cable 1522, which can be coupled to a monitor (not shown) for viewing captured images. The video cable 1522 can be configured to extend from the exterior of the handle's housing, as shown in FIG. 15.

In some embodiments, the shaft 1510 includes the wiring for connecting the camera/illumination unit to the PCB disposed in the handle 1506 and/or to a power supply. The wiring is disposed inside various lumens provided in the shaft 1510. An exemplary arrangement of the shaft is discussed above with regard to FIGS. 1-12.

The tip 1508 includes a flexible portion 1532 and an inflexible portion 1534. In some embodiments, the inflexible portion 1534 can be configured to be a housing for camera and the illumination unit. The flexible portion 1532 is configured to allow bending of the tip 1508. In some embodiments, the tip 1508 can be configured to be connected to a plunger portion 1528 via a push wire 1642 that is further configured to be disposed within the shaft 1510. Upon actuation of the plunger portion 1528, the flexible portion 1532 of the tip 1508 is configured to tilt in such fashion as to allow at least a portion of the tip 1508 to be disposed at an angle with respect to axis of the shaft 1510. Such angular tilting allows view of the cavity from various angles.

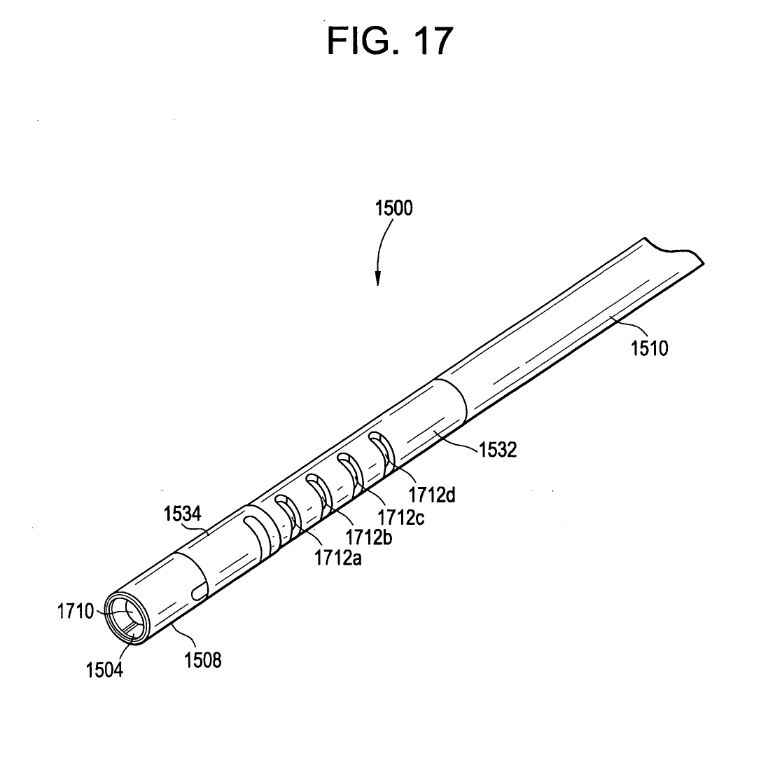

FIG. 17 illustrates tip 1508 in further detail. The tip's 1508 flexible portion 1532 includes partially grooved portions 1712 (a, b, c, d). The grooved portions 1712 are configured to articulate the tip 1508. To articulate the tip 1508, the plunger portion 1528 is actuated, for example, by pressing upon it toward the housing portion of the handle 1506. Upon actuation of the plunger portion 1528, the push wire disposed inside the handle 1506 and the shaft 1510 is configured to push on the tip 1508. Upon pushing the tip 1508, the push wire applies force on the tip 1508, thereby causing the tip 1508 to bend along the grooved portions 1712. Because the grooved portions 1712 are cut through only a portion of the flexible portion 1532, the bending of the tip is permitted in a particular direction, i.e., it allows the user of the device 1500 to control movement of the tip 1508, once the device is inserted into the cavity (i.e., trachea). Further, the upon pushing the tip 1508 (using the plunger portion 1528), the user is also able to control the bending angle of flexible portion 1532. In some embodiments, the flexible portion 1532 can have any range of such angular motion. In some embodiments, the range of motion can be greater than 0 degrees and less than 90 degrees.

In some embodiments, the bending can occur about 1.5 inches before the distal portion of the tip 1508. The grooves 1712 are configured to allow repeatable articulation of the tip. Hence, the user (a doctor or any other medical professional) of the device can potentially re-use the device or vary bending angles of the flexible portion 1532 during examination. The push wire is configured to be anchored proximally to the plunger portion 1528 in the handle 1506, and distally in the tip 1508 that houses the camera. In some embodiments, the bending points (i.e., the grooves 1712) and direction of bending are created by weakening the shaft 1510 of the device 1500 at a specific spot. In some embodiments, the flexible portion 1532 is configured to be the weakening point that allows bending of the tip 1508. When the plunger portion 1528 is depressed, the push wire moves linearly toward the distal end 1504 of the device 1500. As stated above, upon linearly pushing on the distal end 1504 of the tip 1508, the push wire causes the tip 1508 to articulate along the grooved portions 1712. As can be understood by one skilled in the art, the grooved portions 1712 can be cut in the flexible portion 1532 according to various patterns. Further, there can be any number of the grooved portions 1712 disposed in the flexible portion 1532.

In some embodiments, the device 1500 includes a limiter or a holder that is configured to prevent the push wire from bending in an undesirable location, for example inside the handle 1506. Bending of the wire inside the handle 1506 reduces its ability to effectively move in a linear fashion as well as adequately push on the tip 1508. As such, the limiter or the holder that is disposed within the handle 1506 can be configured to limit motion of the push wire inside the handle and confine its motion to a linear axis and prevent its motion in various other directions. In some embodiments, to constrain movement of the push wire inside the shaft 1510, the wire is disposed inside a stiff jacket. The jacket is configured to constrain movement of the push wire within the shaft 1510. In some embodiments, the jacket can be an extruded plastic jacket. As can be understood by one skilled in the art, there can be other ways of limiting unwanted movement of the push wire inside the shaft 1510.

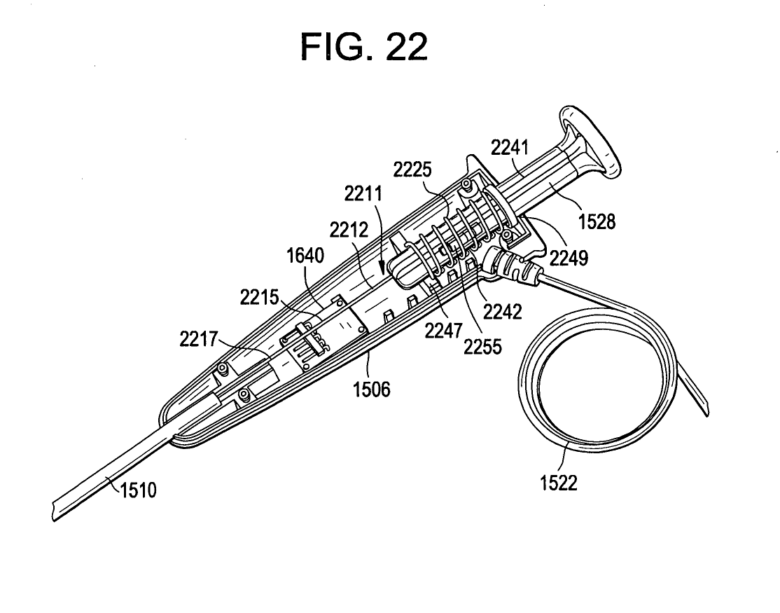

FIG. 22 illustrates a motion-limiting mechanism for constraining motion of the push wire within the handle 1506, according to some embodiments of the present invention. The motion-limiting mechanism includes an extrusion/tube 2212 coupled to the plunger portion 1528 on one end, and is configured to enclose a proximal end 2215 of the stiff jacket 2217 on the other end. The stiff jacket 2217 is configured to be disposed within the shaft 1510, which partially protrudes into the handle 1506, as shown in FIG. 22. The push wire is coupled to the distal end 2211 of the handle 1506 and is configured to be disposed within the stiff jacket 2217 and the tube 2212. Thus, the push wire is configured to be disposed within an enclosed area (i.e., a combination of the stiff jacket 2217 and the tube 2212). Therefore, the push wire is constrained from movement in any direction, except in a linear fashion along the shaft 1510. As such, the push wire does not bend. In some embodiments, the extrusion/tube 2212 can be configured to be removably coupled to the stiff jacket 2217 so that the tube 2212 can move in and out over the stiff jacket 2217 as needed. Thus, upon actuation of the plunger portion 1528, the push wire (anchored at an anchor 1741 disposed at the tip 1508, as shown in FIG. 17) is configured bend the flexible portion 1532 of the tip 1508 with the assistance of the grooved portions 1712.

Referring back to FIG. 22, the plunger portion 1528 of the handle 1506 is a spring-loaded mechanism that upon compression of the spring 2225 is configured to push the push wire toward the tip 1508. The plunger portion 1528 includes a plunger handle 2241, a plunger channel 2242, and the spring 2225. The plunger channel 2242 is configured to be disposed inside the handle 1506 housing and is configured to constrain motion of the plunger handle 2241 in various directions, other than a linear back and forth direction (e.g., along a longitudinal axis of the handle 1506 housing). The spring 2225 is disposed inside the housing of the handle 1506 and is further disposed between stopper points 2247 and 2249, which prevent dislocation or popping-out of the spring 2225 from plunger channel 2242. As such, the spring 2225 is configured to compress and decompress inside the channel 2242.

Upon decompression of the spring 2225, the spring 2225 causes the plunger portion 1528 to extend out and away from the housing of the handle 1506. In some embodiments, in order to prevent over pushing of the push wire, and thus, damaging the tip 1508, the movement of the plunger portion 1528 can be constrained in a way so that the plunger portion 1528 moves only enough to articulate the tip 1508, but not enough to permanently damage it. Over-pushing the tip 1508 can push the push wire too far beyond the limits of the tip, and thus, bent the push-wire irreparably (e.g., to the point of breaking).

In some embodiments, the handle 1506 includes at least one plunger handle limiting device (e.g., a spacer-block) 2255 that is configured to interfere with the motion of the plunger handle 2241. In some embodiments, there can be a plurality of spacer blocks 2255 disposed within the handle 1506. The spacer blocks 2255 can be configured to have different sizes and can be installed in any desired fashion, thus, allowing a customizable installation and movement of the push wire with respect to the tip 1508 and the handle 1506. During manufacturing of the device 1500, the spacer blocks 2255 can be adjusted to provide a proper limit on the motion of the plunger handle 2241 and thus, the push wire.

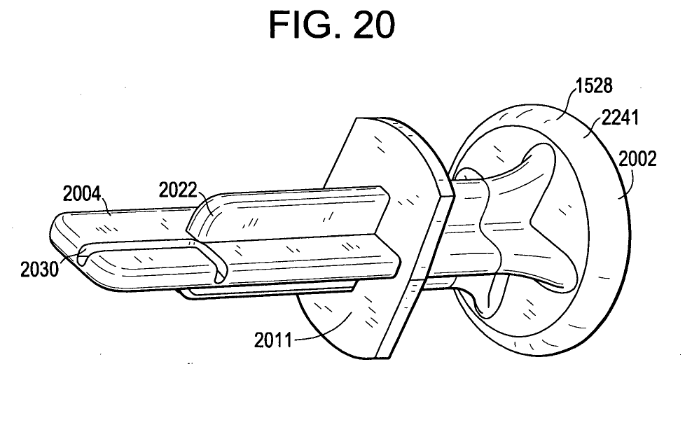

Referring to FIG. 20, the plunger handle 2241 is configured to include an actuation tip 2002 coupled to a shaft 2004. The shaft 2004 includes a stopper surface 2011 that prevents the handle 2241 from popping out from the housing of the handle portion 1506 (not shown). The shaft 2004 further includes a groove 2022 that is configured to interact with spacer blocks 2255 (not shown) and prevent further displacement of the handle 2241 into the housing of the handle portion 1506. The shaft 2004 also includes a channel 2030 for securing the push wire 1642. The push wire 1642 can be configured to be protracted along the channel 2030 and then secured to the shaft 2004.

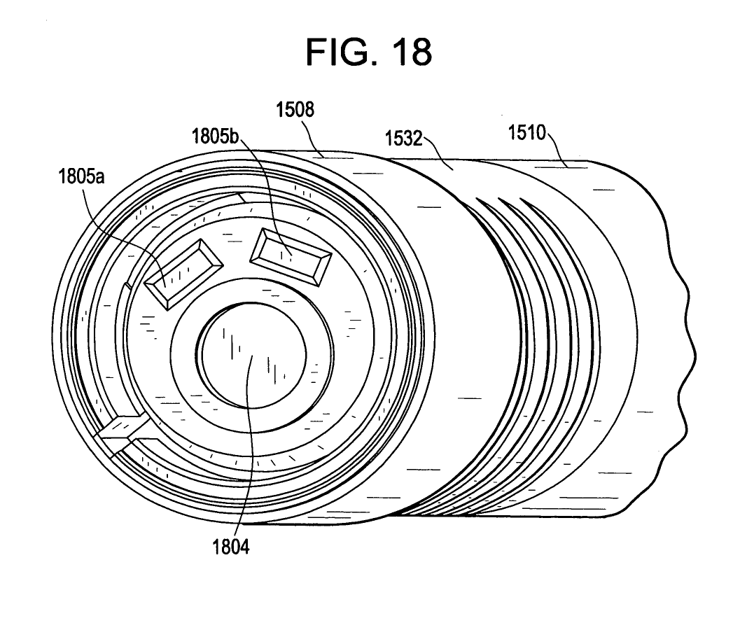

FIG. 18 is a front view of the tip 1508 having a camera 1804 and illumination units 1805 (a, b). The camera 1804 can be disposed behind a protective cover. The illumination units 1805 are configured to illuminate the path and/or the cavity to be examined so that the camera 1804 can capture images and transfer them to a monitor coupled to device 1500.

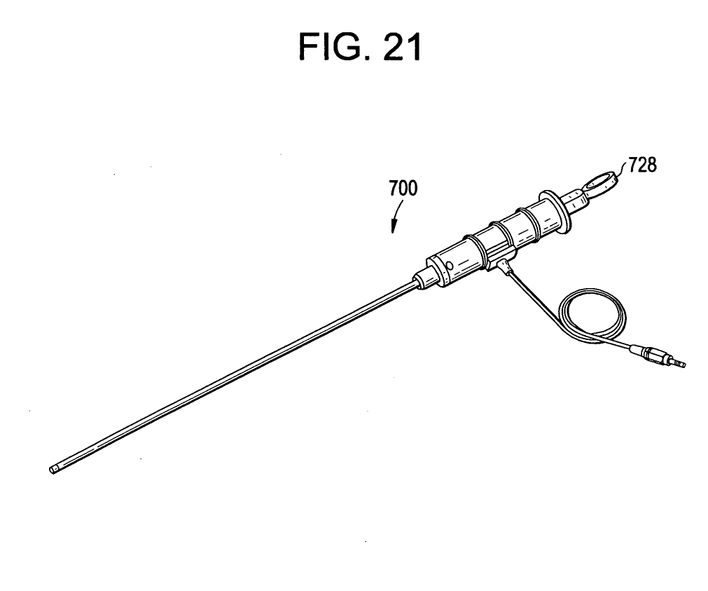

FIG. 21 illustrates an alternate embodiment of an intubation device 2100, according to some embodiments of the present invention. The intubation device 2100 includes a ring-type handle portion 2128 that allows a user to insert his/her finger into the ring and push/pull on the handle during actuation of the tip.

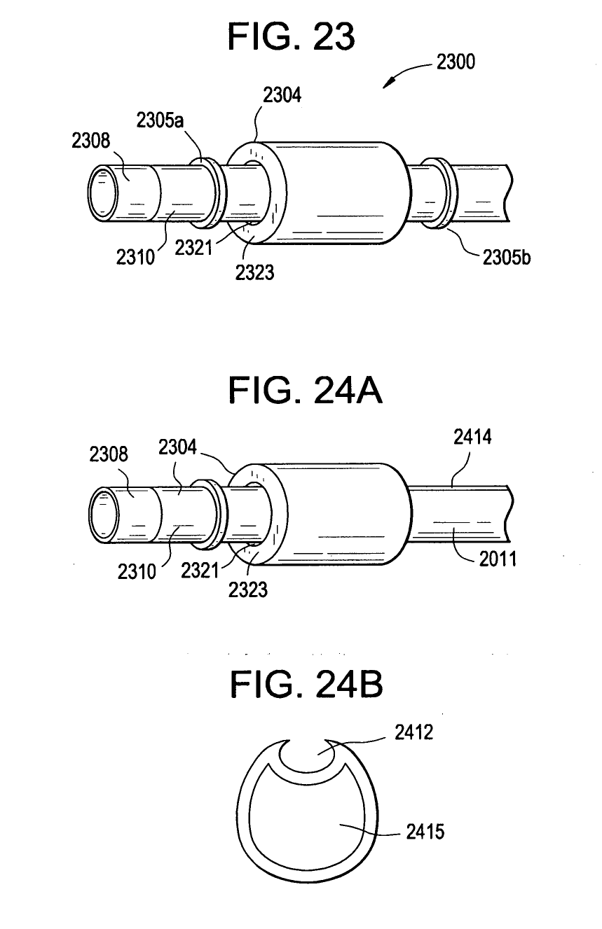

FIGS. 23-24 illustrate another alternate embodiment of the intubation device 2300, according to some embodiments of the present invention. In some embodiments, the users (doctors, anesthesiologists, or other medical professionals) may desire to dilate certain parts of patient's anatomy (e.g., portion of patient's trachea) in order to implant a tube that is bigger than the opening into which it is implanted. Conventional procedures for doing so (for example, in intensive care units) include having intensivists put tracheostomies percutaneously into patients on a long-term ventilation. This involves putting a wire into the trachea and gradually dilating up the hole by a series of bougies until a tracheostomy tube can be put in. All of these patients have an oral tube already inserted. In order to check that the wire has been placed in the right part of the trachea a bronchoscope is put down into the oral tube. In some cases, an intubation device could be used instead of this, because it is much easier to use and requires much less equipment than a bronchoscope.

To alleviate the drawbacks associated with conventional procedures, the present invention includes an inflatable circular balloon structure 2304 that can be put around the shaft 2310 of the intubation device 2300. The balloon structure 2304 can be configured to be inflated and thus, expand the patient's trachea without a need to swap out different bougies. In some embodiments, the shaft 2310 is coupled to a tip 2308 that includes a camera and/or an illumination device. The shaft 2310 further includes stop features 2305 (a, b) disposed on the shaft and substantially adjacent the tip portion. The stop features 2305 can be ring-like structures that are wrapped around the shaft 2310. The balloon structure 2304 is configured to be disposed on the shaft 2310 between the stop features 2305. The balloon structure 2304 can be configured to include a tube portion 2321 and a balloon portion 2323 that is disposed within the tube portion 2321. The tube portion 2321 is configured to slide over the shaft 2310. Upon insertion of the device 2300 down the patient's trachea (or any other cavity), the balloon portion 2323 is configured to be inflated, thereby increasing the size of the balloon structure 2304 and hence dilating the patient's trachea. The amount of inflation of the balloon portion 2323 controls the amount of dilation of the patient's trachea. While the patient's trachea is dilated or dilating the tracheal opening, the visualization part (i.e., the tip 1508 containing the camera/illumination units) can be moved back and forth since the tube portion 2321 is configured to slide between the stop features 2305. In some embodiments, the tube portion 2321 can include a hardened surface that is placed around the shaft 2310 to prevent friction between the tube portion 2321 and the shaft 2310 as well as overexpansion of the tube portion 2321 during inflation of the balloon portion 2323. Further, the shaft 2310 and the tube portion 2323 can be manufactured from a biocompatible, substantially frictionless material to prevent unnecessary stoppage of the balloon structure 2304 during dilation. In some embodiments, the balloon structure 2304 can be fixed to the shaft 2310. As can be understood by one skilled in the art, the balloon structure 2304 can be a unitary structure that allows inflation of the structure 2304 via an outside air hose. (not shown in FIG. 23).

In some embodiments, the balloon structure 2304 can be fixed onto the shaft 2310 at a predetermined location. For example, the balloon structure 2304 can be configured to be fixed closer to the tip 1508 or further away from it. Additionally, the stop features can be further spaced apart, thus, allowing the user greater degree of freedom (i.e., linear movement). In some embodiments, the balloon structure could be allowed to slide back and forth on the shaft but with stop features to limit its motion to a certain range only. The balloon inflation can be configured to be controlled from the proximal end of the device (for example, using a hand pump (e.g., similar to those used to inflate blood pressure cuffs)). In some embodiments, the balloon structure 2304 can include an air tube lumen 2412 through which air can be pumped into the balloon structure 2304 via an air tube 2414, as shown in FIG. 24. The air is configured to travel from a pump (not shown) along the tube 2414 that can be embedded inside an open channel in the shaft 2310, as shown in FIG. 24. In some embodiments, the air tube lumen 2412 can be configured to be coupled to the open channel in the shaft 2310 (such as via snap-in features, or any other type connection devices). Upon connection to the open channel in the shaft 2310, the air tube lumen is configured to slide back and forth. In some embodiments, the connection between the air tube that inflates the balloon and the balloon can be disposed underneath the balloon. In alternate embodiments, such connection can be accomplished via an unattached tube, or in the case of the fixed location balloon embodiment, a tube that is fixed to the outside or built inside the main shaft of the device.

In some embodiments, the distal end of the intubation device can be washed with saline or any other washing liquid. The camera can be moved back and forth inside the housing of the tip of the device. The housing of the tip can be filled with washing liquid, and may also include a diaphragm having a material to “wipe” the lens. Accordingly, the camera can be washed by rotating, spinning, or any other motion that allows cleaning of the camera lens using the diaphragm, with or without the use of the washing liquid (e.g., saline). Once washed, the camera can be pushed back toward the end of the tip.

In some embodiments, the present invention can include an electronic means, configured to disable the device after a certain number of uses, where a “use” can be defined as turning the device from the OFF state to the ON state. In some embodiments, a non-volatile memory can be incorporated in the PCB that stores these “uses”. When device is powered up, the microcontroller reads a stored value of “uses” in the memory. In some embodiments, such memory can be a use-counter configured to increment number of “uses” after each use. Thus, after powering up, the microcontroller increments the use-counter and then stores the value to the non-volatile memory (in some embodiments, the new “use” value replaces the old “use” value). After the counter reaches a predetermined number of uses (e.g., a threshold “use” value), a signal can be generated informing the microcontroller (or any other component) to disable or turn off the device.

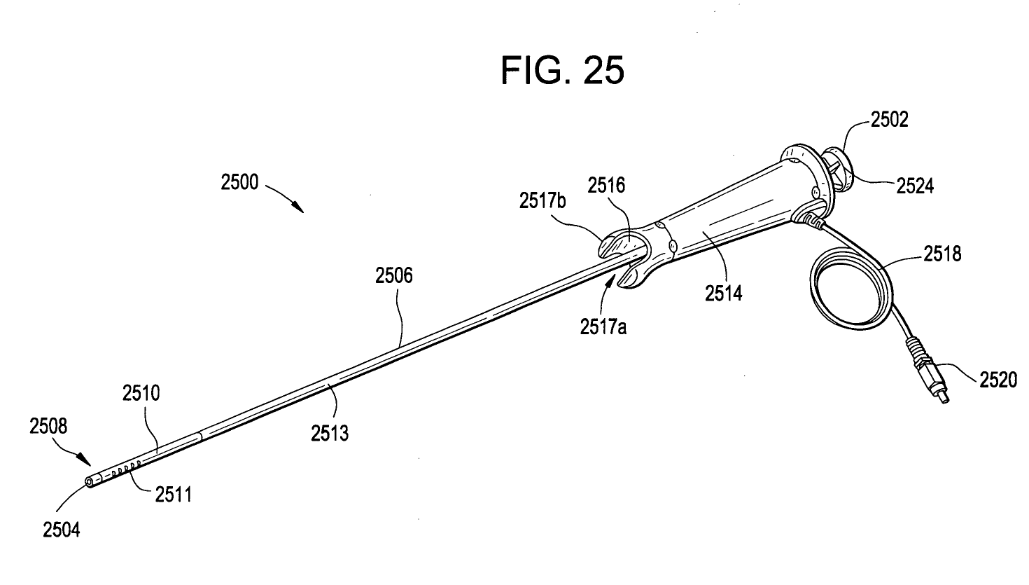

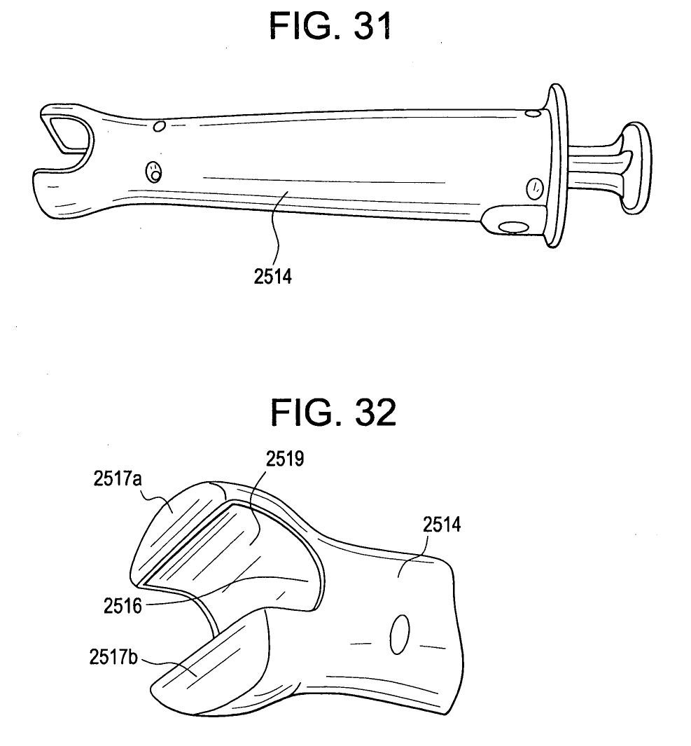

FIGS. 25-32 illustrate another exemplary embodiment of an intubation device 2500, according to some embodiments of the present invention. The device 2500 includes a handle 2514 disposed at a proximate end 2502 and a tip 2508 having a camera (not shown) disposed at a distal end 2504. A shaft 2506 is disposed between the handle 2514 and the distal end 2504. The shaft includes a rigid portion 2513 and a flexible portion 2510. The rigid portion 2513 is configured to be coupled to the handle 2514 and the flexible portion 2510 is configured to be disposed substantially adjacent to the distal end 2504 and further configured to be coupled to the tip 2508. The flexible portion 2510 is configured to include a plurality of indentations 2511 (which are similar to the grooved portions 1712 shown in and discussed in connection with FIG. 17 above). The flexible portion 2510 is configured to bend thereby articulating the tip 2508 in a desired direction. Similar to the grooved portions 1712, the indentations 2511 are configured to allow the tip 2508 to articulate between 0 and 90 degrees. In some embodiments, the indentations 2511 are configured to allow articulation of tip 2508 in various directions. The indentations 2511 are configured to allow multiple articulations of the tip 2508, thus, allowing the device 2500 to be used multiple times.

The shaft 2506 is configured to contain a push wire, a video/power/data cable(s), various channels for transporting of tools/flushing liquids, etc. (not shown in FIG. 25). These components are similar to the like components discussed above with regard to FIGS. 1-24, and as such, will not be discussed again.

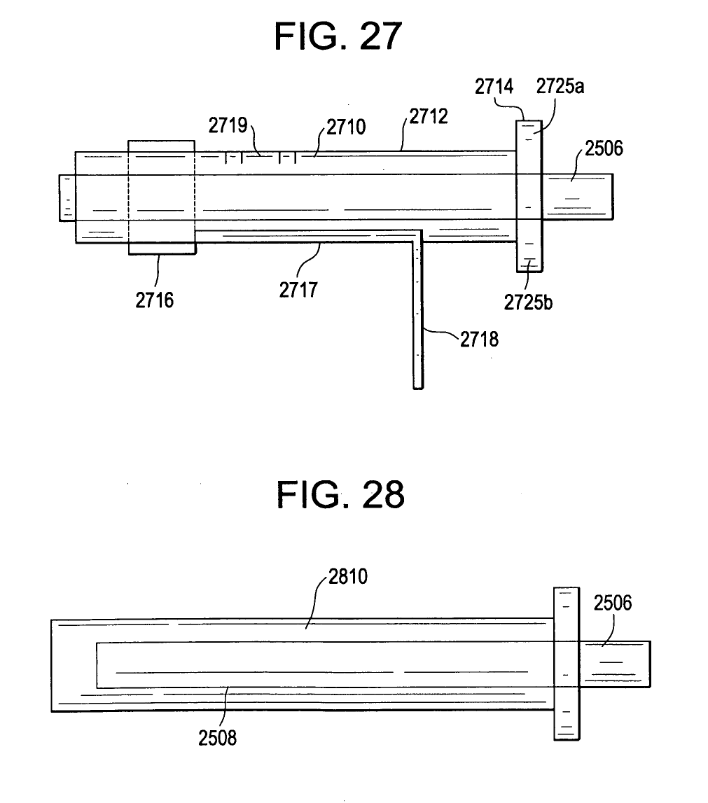

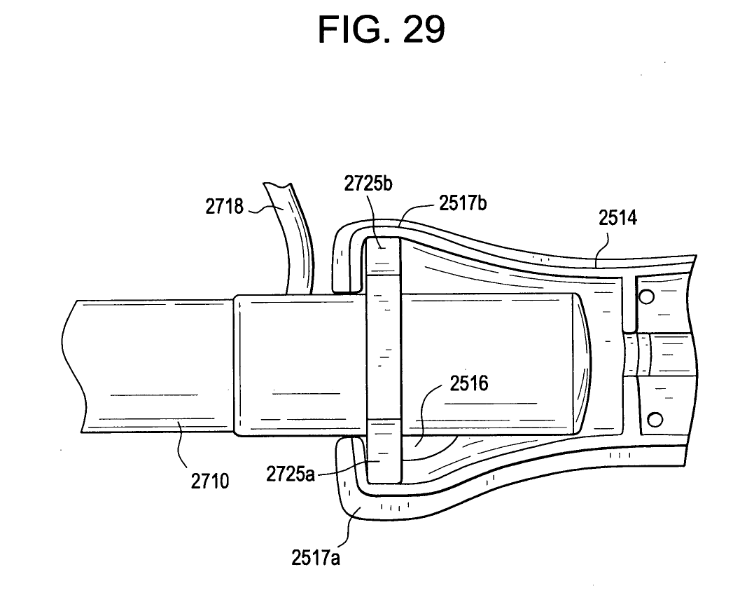

The handle 2514 further includes an actuation handle (or a plunger handle) 2524 that is configured to be coupled to a push wire disposed inside the handle 2514 and further coupled to the tip 2508. Upon depressing the actuation handle 2524 toward the housing of the handle 2514, the tip 2508 is configured to articulate. When the handle 2524 is released, the tip 2508 is configured to return to its original location. The handle 2524 is further coupled to a wiring 2518 having an adaptor 2520, which can be used to connect the device 2500 to a power/processing equipment/monitor (not shown in FIG. 25). The handle 2514 further includes an enclosure section 2516 having two protruding sections (or protrusions) 2517a and 2517b. The enclosure section 2516 is disposed distal to the proximal end 2502. The enclosure section 2516 is configured to retain an insertion depth measuring tube (shown in FIGS. 27 and 29). In some embodiments, the distance between the protrusions 2517 is configured to be smaller than the diameter of the enclosure section 2516.

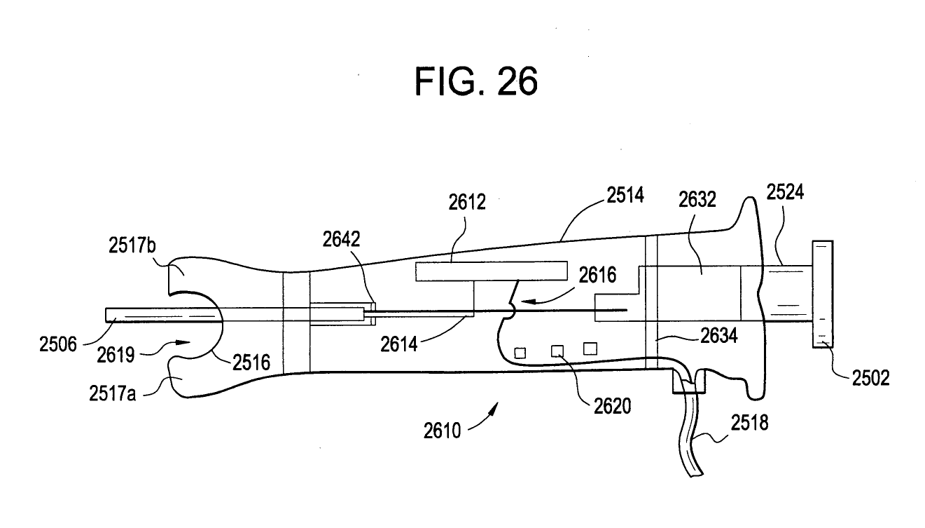

FIG. 26 is a cross-sectional view of the handle 2514 of the device 2500 shown in FIG. 26. The handle 2514 has a housing 2610 that is configured to enclose various components of the device 2510. The housing 2610 includes a PCB 2612 (similar to the PCBs of the devices shown and described with regard to FIGS. 1-24) configured to transmit and receive power/video/data to and from the camera disposed in the tip 2508. The PCB 2612 is coupled to the camera (not shown in FIG. 26) via a power/video/data wire/cable 2614. As can be understood by one skilled in the art, there can be more than one power/video/data wire/cable 2614 (e.g., one for each of power, data, and video). The PCB 2612 is further coupled to an external power supply/processing equipment/monitor (not shown in FIG. 26) via a wiring 2620. In some embodiments, there can be more than wiring 2620. Further, in some embodiments, the PCB 2612 can wirelessly communicate with external power/supply/processing equipment/monitor. In this case, the PCB 2612 can also be configured to include a power source to power its operation and the operation of the camera and lighting equipment disposed inside the tip 2508 (not shown in FIG. 26).

The actuation handle 2524 is coupled to a push wire 2616 that is configured to protrude through the entire housing of the handle 2514 and into the shaft 2506 (a portion of which is disposed inside the handle housing 2610). Upon actuation (i.e., depressing on) of the handle 2524, the push wire 2616 is configured to be forced toward the tip 2508 (not shown in FIG. 26) and cause articulation of the tip 2508. The handle 2524 is configured to include a spring loaded mechanism 2632 (e.g., a spring) that is further configured to be disposed between a stopper 2634 and the housing 2612. The spring-loaded mechanism 2632 is configured to push back on the handle 2524, thereby forcing the handle 2524 to return to its pre-depressed state and thus, causing the tip 2508 to return to its pre-articulated position.

The handle 2514 further includes an encasing 2642 for securing of the shaft 2506. The encasing 2642 is configured to prevent the shaft 2506 from moving with along with the push wire 2616, when the later is pushed by the handle 2524. In some embodiments, the push wire 2616 is configured to be enclosed in a channel that prevents the push wire 2616 from kinking or bending while inside the handle 2514 (similar to what has been discussed above with regard to FIG. 22).

As shown in FIGS. 25-26 and 30-31, the protrusions 2517 are configured to be disposed opposite one another, thereby creating two oppositely disposed openings 2619 between protrusions 2517 that are disposed in the enclosure section 2516. The openings 2619 are configured to allow insertion of an endo-tracheal insertion tube (shown in FIG. 27), whereby protrusions 2517 are configured to lock the endo-tracheal tube, when the latter is rotated into a locking position (shown in FIG. 29). Endo-tracheal tubes are conventionally known and used by medical professionals during intubation of patient and ensure that that patient's airway is not closed off during intubation and that air is able to reach the lungs.

FIG. 27 illustrates an exemplary endo-tracheal tube 2710 that is configured to be placed over the shaft 2506, according to some embodiments of the present invention. The tube 2710 includes a hollow housing 2712 coupled to a stopper mechanism or a locking feature 2714, having oppositely disposed locking tabs 2725(a, b), disposed at a proximal end of the housing 2712. The stopper 2714 is configured to have a larger diameter than the diameter of the housing 2712. In some embodiments, the diameter of the housing 2712 is configured to be slightly larger than the diameter of the shaft 2506 so that the housing 2712 can be placed over the shaft 2506. In some embodiments, the housing 2712 can further include depth marks 2719 that can be disposed along the entire length of the housing 2712. The marks 2719 can be configured to allow the user to determine the depth of insertion of the tube 2710. The tube 2710 further includes an inflatable balloon or cuff 2716. The balloon 2716 is configured to be disposed substantially adjacent the distal end of the housing 2712. In some embodiments, the balloon 2716 is configured to be disposed around a portion of an outer perimeter of the housing 2712, as shown in FIG. 27. The balloon 2716 is further coupled to an air channel 2717 that connects to an inflation hose 2718. The hose 2718 can be coupled to a pump for pumping air into the balloon 2716. The channel 2717 is configured to be disposed within the wall of the housing 2712 so that it does not interfere with the insertion of the shaft 2506 into the tube 2710. Upon insertion of the tube 2710 into the trachea of the patient at a particular depth, the user (e.g., a doctor, or any other medical professional) inflates the balloon 2716 using an air pump (not shown in FIG. 27) so that the balloon 2716 extends to the interior wall of the trachea of the patient, thereby constricting any further movement of the tube 2710 down the trachea of the patient. The shaft 2506 can then be moved inside the housing 2710, so that the user can visualize the interior of the trachea of the patient.

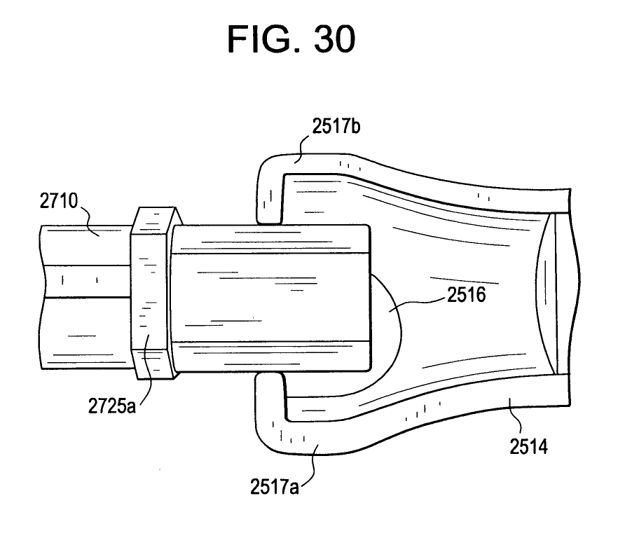

In some embodiments, the shaft 2506 can be interlocked with the tube 2710 using the locking feature 2714, whose locking tabs 2725 are configured to interact with the protrusions 2517, as shown in FIG. 29. FIG. 29 illustrates the locking feature 2714 being inserted into the enclosure section 2516 of the handle housing 2610 and the protrusions 2517 holding the locking tabs 2725 inside the enclosure section 2516. When the tube's locking feature 2714 is configured to be interlocked with the enclosure section 2516, the device 2500 and the tube 2710 are configured to move together inside the trachea of the patient. To unlock the device 2500 from the tube 2710, the device 2500, using the handle 2514 is rotated until the protrusions 2517 are free from the locking tabs 2527, i.e., the locking tabs 2527 are configured to be inside the openings 2619, as shown in FIG. 30. Upon such rotation, the device 2500 is free from the tube 2710, and can be moved back and forth (as well as can be removed from the patient).

To remove the tube 2710 from the patient, the balloon 2716 is deflated via channel 2717 and the hose 2718 and the tube 2710 is removed. In some embodiments, the tube 2710 can be removed using the device 2500 by interlocking the protrusions 2517 with the locking tabs 2725 of the tube 2710 and then pulling the tube 2710 along with the device 2500 out of the patient. In some embodiments, the tube 2710 can be configured to be shorter than the shaft 2506, so that the shaft 2506 can be protruded outside the distal end of the tube 2710, but not beyond the stopper feature 2714. In some embodiments, the markings 2719 can be configured to correspond to the maximum insertion depth of the shaft 2506. In some embodiments, the tube 2710 can be configured to be rigid to prevent flexing of the tube during insertion into the patient.

FIG. 28 illustrates an exemplary directional tube 2810, according to some embodiments of the present invention. The directional tube 2810 can be configured to be placed over the shaft 2506 in a similar fashion as the tube 2710 shown in FIG. 27. The tube 2810 can be configured to be flexible and can flex together with the flexible portion of the shaft 2506. Upon insertion of the tube 2810 that contains the shaft 2506 into the patient to a predetermined depth, the user (e.g., a doctor, or any other medical professional) can articulate the tip 2508 and determine the direction of insertion. Upon determining the proper direction of insertion, the shaft 2506 can be further inserted along the requisite direction.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. An intubation guidance device for guiding insertion of a intra-trachea tube into a patient, comprising:
a handle disposed at a proximal end of the intubation device;
a guiding tip having a camera disposed at a distal end of the intubation device;
an inter-trachea tube connecting said handle and said guiding tip;
said camera is configured to guide insertion of an intra-trachea tube into a patient, wherein said guiding tip is configured to be inserted inside the intra-trachea tube while said handle remains outside the intra-trachea tube.

2. The device according to claim 1, wherein said inter-trachea tube further comprises a plurality of protrusions disposed on an outside surface of said inter-trachea tube and configured to extend away from said outside surface to secure said inter-trachea tube inside the intra-trachea tube.

3. The device according to claim 1, wherein said handle includes a handle housing configured to enclose a handle printed circuit board (“handle PCB”) configured to process and supply power to and from said camera.

4. The device according to claim 3, wherein said tip further comprises a tip housing configured to enclose
a tip printed circuit board (“tip PCB”) configured to be connected to said camera and further configured to process and supply signals to and from said camera and to and from said handle PCB; and
said camera.

5. The device according to claim 4, wherein said inter-trachea tube further comprises:
a wire bundle having
a power/data wire configured to connect said handle PCB and said tip PCB;
a stiffening wire configured to provide stiffness to said inter-trachea tube when said inter-trachea tube is being inserted into an intra-trachea tube of the patient;
a bendable stylette configured to allow the inter-trachea tube to be bent and secured inside the intra-trachea tube; and
a lumen for insertion of a pull wire, wherein said pull wire is configured to assist in guiding said inter-trachea tube.

6. The device according to claim 5, wherein said tip housing further includes a protective lens configured to protect said camera from damage.

7. The device according to claim 6, wherein said camera is configured to move inside said tip housing to allow cleaning of a lens of said camera such that the camera lens optionally moves through a diaphragm.

8. An intubation guidance device for guiding insertion of an intra-trachea tube into a patient, comprising:
a handle disposed at a proximal end of the intubation device;
a guiding tip having a camera disposed at a distal end of the intubation device;
a wire bundle connecting said handle and said guiding tip;
said camera is configured to guide insertion of an intra-trachea tube into a patient, wherein said guiding tip is configured to be inserted inside the intra-trachea tube while said handle remains outside the intra-trachea tube.

9. The device according to claim 8, wherein said wire bundle further comprises a plurality of protrusions disposed on an outside surface of said wire bundle and configured to extend away from said outside surface to secure said wire bundle inside the intra-trachea tube.

10. The device according to claim 8, wherein said handle includes a handle housing configured to enclose a handle printed circuit board (“handle PCB”) configured to process and supply power to and from said camera.

11. The device according to claim 10, wherein said tip further comprises a tip housing configured to enclose
a tip printed circuit board (“tip PCB”) configured to be connected to said camera and further configured to process and supply signals to and from said camera and to and from said handle PCB; and
said camera.

12. The device according to claim 11, wherein said wire bundle further comprises:
a power/data wire configured to connect said handle PCB and said tip PCB;
a stiffening wire configured to provide stiffness to said wire bundle when said wire bundle is being inserted down the intra-trachea tube of the patient.

13. The device according to claim 12, wherein said tip housing further includes a protective lens configured to protect said camera from damage.

14. The device according to claim 13, wherein said camera is configured to move inside said tip housing to allow washing of a lens of said camera.

15. An intubation guidance device for guiding insertion of a intra-trachea tube into a patient, comprising:
a handle disposed at a proximal end of the intubation device;
a guiding tip having a camera disposed at a distal end of the intubation device;
a malleable metal tube connecting said handle and said guiding tip;
said camera is configured to guide insertion of an intra-trachea tube into a patient, wherein said guiding tip is configured to be inserted inside the intra-trachea tube while said handle remains outside the intra-trachea tube.

16. The device according to claim 15, wherein said metal tube further comprises a plurality of protrusions disposed on an outside surface of said metal tube and configured to extend away from said outside surface to secure said metal tube inside the intra-trachea tube of the patient.

17. The device according to claim 15, wherein said handle includes a handle housing configured to enclose a handle printed circuit board (“handle PCB”) configured to process and supply power to and from said camera.

18. The device according to claim 17, wherein said tip further comprises a tip housing configured to enclose
a tip printed circuit board (“tip PCB”) configured to be connected to said camera and further configured to process and supply signals to and from said camera and to and from said handle PCB; and
said camera.

19. The device according to claim 18, wherein said metal tube further comprises:
a power/data wire configured to connect said handle PCB and said tip PCB.

20. The device according to claim 19, wherein said tip housing further configured to be inserted inside said metal tube and further includes a protective lens configured to protect said camera from damage.

21. The device according to claim 20, wherein said camera is configured to move inside said tip housing to allow washing of a lens of said camera.

22. The device according to claim 21, further comprising a use counter configured to count a number of times the device is powered up from an off-state to an on-state;
the device is configured to be disabled after said number of times is equal to a predetermined number of times the device can be powered up from said off-state to said on-state.

23. An intubation device for examination of a patient, comprising:
a handle disposed at a proximal end of the intubation device;
a guiding tip having a camera disposed at a distal end of the intubation device;
a shaft portion connecting said handle and said guiding tip;
said camera is configured to guide insertion of said shaft portion into a patient, wherein said guiding tip is configured to be inserted inside said shaft portion while said handle remains outside the patient;
said shaft portion includes an articulation mechanism configured to angularly articulate position said guiding tip with respect to said shaft.

24. The device according to claim 22, wherein said handle includes an articulation handle configured to be coupled to said guiding tip using a push wire, wherein upon actuation of said articulation handle, said articulation mechanism is configured to angularly articulate position of said guiding tip.

25. The device according to claim 22, wherein said handle includes a handle housing configured to enclose a handle printed circuit board (“handle PCB”) configured to process and supply power to and from said camera.

26. The device according to claim 25, wherein said tip further comprises a tip housing configured to enclose
said camera for capturing images; and,
an illumination unit configured to illuminate a field of view of said camera.

27. The device according to claim 26, wherein said tip housing further includes a protective lens configured to protect said camera from damage.

28. The device according to claim 24, wherein said shaft portion further comprises:
a power/data wire configured to connect said handle PCB and said camera/illumination unit; and,
a stiffening jacket configured to constrain movement of said push wire to a movement along an axis of said shaft portion and to prevent unwanted bending of said push wire.

29. The device according to claim 28, wherein said handle housing further comprises slidable tube containing said push wire and further configured to constrain movement of said push wire to a linear movement;
wherein said slidable tube is coupled to said actuation handle and slidably coupled to said stiffening jacket;
wherein said stiffening jacket is configured to partially protrude inside said handle housing.

30. The device according to claim 29, wherein said handle housing further includes a stop feature configured to interact with said actuation handle to prevent said handle from over-pushing said push wire inside said shaft portion.

31. The device according to claim 30, wherein said stop feature is adjustable.

32. The device according to claim 23, wherein said articulation mechanism includes a plurality of grooves disposed on said shaft portion substantially adjacent to said guiding tip, thereby creating a weakening portion of shaft that allows articulation of said guiding tip.

33. The device according to claim 23, wherein said articulation mechanism is configured to allow a variable angle of articulation.

34. The device according to claim 33, wherein said angle of articulation is in a range of 0 degree to 90 degrees.

35. The device according to claim 23, wherein said camera is configured to move inside said tip housing to allow washing of a lens of said camera.

36. The device according to claim 35, further comprising a use counter configured to count a number of times the device is powered up from an off-state to an on-state;
the device is configured to be disabled after said number of times is equal to a predetermined number of times the device can be powered up from said off-state to said on-state.

37. An intubation device for examination of a patient, comprising:
a handle disposed at a proximal end of the intubation device;
a guiding tip having a camera disposed at a distal end of the intubation device;
a shaft portion connecting said handle and said guiding tip;
said camera is configured to guide insertion of said shaft portion into a patient, wherein said guiding tip is configured to be inserted inside said shaft portion while said handle remains outside the patient;
said shaft portion includes a dilation mechanism configured to dilate an examination cavity within the patient.

38. The device according to claim 37, wherein said dilation mechanism further comprises an inflatable portion configured to be inflated upon insertion of the guiding tip into the examination cavity within the patient.

39. The device according to claim 38, wherein said inflatable portion is configured to be slidably mounted on said shaft portion;
wherein said shaft and said tip portion are configured to translate along said inflatable portion while said inflatable portion is being inflated.

40. The device according to claim 39, wherein said dilation mechanism includes at least one stop feature disposed on said shaft portion and configured to prevent removal of said inflatable portion from said shaft portion.

41. The device according to claim 49, wherein said inflatable portion is configured to be connected to an air pump configured to pump air into and inflate said inflatable portion.

42. The device according to claim 38, wherein said shaft portion is configured to include an embedded air tube removably coupled to said inflatable portion and configured to supply air to and inflate said inflatable portion.

43. The device according to claim 42, wherein said shaft portion includes a lumen disposed on an outer surface of said shaft for holding said embedded air tube.

44. A method of intubating a patient using an intubation device, having
a handle disposed at a proximal end of the intubation device;
a guiding tip having a camera disposed at a distal end of the intubation device; and
a shaft portion connecting the handle and the guiding tip;
the shaft portion includes an articulation mechanism configured to angularly articulate position the guiding tip with respect to the shaft;
the method comprising the steps of:
using the camera, guiding insertion of the guiding tip and shaft portion into a patient, wherein the guiding tip is configured to be inserted inside the shaft portion while the handle remains outside the patient;
using the articulation mechanism, angularly articulating the guiding tip within the patient; and
using the camera, examining the patient.

45. A method for intubating a patient using an intubation device having
a handle disposed at a proximal end of the intubation device;
a guiding tip having a camera disposed at a distal end of the intubation device;
a shaft portion connecting the handle and the guiding tip; and
the shaft portion includes a dilation mechanism configured to dilate an examination cavity within the patient;
the method comprising the steps of:
using the camera, guiding insertion of the shaft portion into a patient, wherein the guiding tip is configured to be inserted inside the shaft portion while the handle remains outside the patient;
using the dilation mechanism, dilating the examination cavity within the patient; and
using the camera, examining the examination cavity.
